(19) United States
(12) Patent Application Publication
(10) Pub. No.: US 2018/0200392 A1
(43) Pub. Date: Jul. 19, 2018

(54) DETECTION AND TREATMENT OF RENAL CELL CARCINOMA WITH A SLC6A3 LIGAND LINKED TO A LABEL, CYTOTOXIC OR IMMUNOMODULATORY GROUP

(71) Applicant: Akuru Pharma AB, Lund (SE)

(72) Inventors: Håkan Axelson, Lund (SE); Jennifer Hansson, Åkarp (SE); David Lindgren, Malmö (SE); Lena Gustavsson, Lund (SE); Elinn Johansson, Lund (SE)

(21) Appl. No.: 15/743,352

(22) PCT Filed: Jul. 4, 2016
(86) PCT No.: PCT/EP2016/065691

§ 371 Date: Jan. 10, 2018

(30) Foreign Application Priority Data
Jul. 16, 2015 (SE) 1551024-1

(51) Int. Cl. A61K 51/04

(52) U.S. Cl. CPC A61K 51/0448; A61K 51/0459; A61K 51/0455

(57) ABSTRACT

The invention provides methods and material for diagnosis and treatment of ccRCC. Thus, the invention relates to a method for diagnosis or treatment of clear cell renal cell carcinoma (ccRCC) in an individual in an individual in need thereof, wherein the method comprises use of an SLC6A3 ligand linked to a radioactive label, a cytotoxic moiety or an immunomodulatory moiety.

### FIELD OF INVENTION

The present invention relates to materials and methods for detection of clear cell renal cell carcinoma (ccRCC). In particular, the invention relates to use of dopamine transporter ligands for detection of ccRCC.

### BACKGROUND OF INVENTION

Renal cell carcinoma (RCC) accounts for approximately 3% of adult cancer cases and is the second most common urologic neoplasm. In Sweden there are approximately 1000 cases of RCC annually. The main types of RCC are ccRCC, papillary RCC and chromophobe RCC, which have different prognoses and therapeutic indications. ccRCC is by far the most common type, representing approximately 75% of all RCC.

Localized ccRCC tumors have a good prognosis, where the patients undergo nephrectomy (removal of the effected kidney) and the 5 year survival is about 85-90%. However, one third of all patients present with metastasis already at diagnosis. Patients with spread disease have a very dismal prognosis, with a 5 year survival of around 20%. Accordingly, methods for early detection of RCC are desirable.

Because common anti-tumoural therapies like cytostatics and radiation have no, or very limited effect on RCC, the current treatment regimen for metastatic disease is based on inhibition of angiogenesis using small kinase inhibitors such as sorafenib and sunitinib or mTOR inhibitors such as temsirolimus, but resistance development remains a huge problem and the clinical benefits are limited.

Computed tomography (CT) and magnetic resonance (MR) imaging have historically been used to assess tumor response to therapy on the basis of morphologic criteria (RECIST), a classification based on changes in lesion size. Functional imaging using ¹⁸F-FDG-PET (¹⁸F is combined with the glucose analogue 2-fluoro-2-deoxy-D-glucose), is widely used for detection and imaging of solid cancers. The method is based on the observation that tumors are more metabolically active than the surrounding tissues and hence metabolize more sugar. ¹⁸F-FDG-PET has also been used to study RCC and recent reports indicate it potentially can be used to predict progression-free survival of patients treated with TKIs. It should however be pointed out that degree of uptake of ¹⁸F-FDG in RCC is relatively low, which limits the size of detectable lesions. Furthermore, uptake may also be observed during other non-malignant conditions such as chronic inflammation and infections. Furthermore, ¹⁸F-FDG-PET cannot be used for distinction between the different subtypes of RCC. This is becoming increasingly more important as the different subtypes of RCC respond differently to the different treatment modalities now available.

### SUMMARY OF INVENTION

Thus, there is an unmet need for improved detection and treatment modalities for ccRCC. For example methods for detecting ccRCC and/or methods providing distinction between localized and metastasized disease are desirable.

The present invention provides novel methods for detection of ccRCC. The methods of the invention are based on the surprising finding that ccRCC cells display an enhanced uptake of [³H]-dopamine compared to normal kidney cells and to other subtypes of RCC. In the synaptic cleft of neurons, dopamine is transported into cells with the aid of Solute carrier family member 3 (SLC6A3), which is also known as “dopamine transporter”. The present invention demonstrates that ccRCC expresses functional SLC6A3, which can mediate uptake of dopamine, whereas normal kidney do not express SLC6A3 at all. Expression of functional SLC6A3 can be utilised in methods of diagnosis and/or therapy as described herein.

Various array based studies on differential gene expression in RCC have previously indicated that at least fragments of SLC6A3 RNA are expressed at high levels in RCC (see e.g. Skubitz and Skubitz, 2002). However, results from an array based platform, as in Skubitz and Skubitz, merely indicates the presence of a transcript, but does not provide any information on the presence of a functional protein. For example, when using an array based platform there is always a risk that the probe cross-react with other transcripts, especially in the case of large homologous gene families such as the solute carrier family, which comprise more than 300 members. Furthermore, when using array-based platforms it is impossible to know whether the expressed transcript is expressed in its functional form, e.g. that the transcript is expressed in an alternate splice form or a consequence of erroneous initiation of a non-functional transcript that lacks the capacity to code for a protein (Chen and Weiss, Oncogene 2015). There are numerous reports of such tumor-specific aberrant transcripts. In addition, it is widely established that mRNAs can regulate gene translation without affecting mRNA levels. Thus, high mRNA levels do not necessarily correlate with elevated protein levels, and/or elevated protein function.

SLC6A3 mediates reuptake of dopamine from the synaptic cleft of neurons. The function of SLC6A3 is highly dependent on a Na⁺ gradient, with high extracellular concentration (Torres et al., 2003). Two Na⁺ ions and one Cl⁻ ion bind SLC6A3 and induce a conformational change where the ions are co-transported with one molecule of dopamine into the intracellular space (Sonders et al., 1997). The Na⁺ gradient is achieved through the effect of plasma membrane Na⁺/K⁺ ATPases. Lower Na⁺ concentration mediates decrease uptake of [³H]-dopamine as well as a decrease in maximal velocity of the uptake in vitro (Wheeler et al., 1993). Thus, it has been believed that the microenvironment of the synaptic cleft is essential for the function of SLC6A3 protein as dopamine transporter.

The present invention surprisingly demonstrates that even in the microenvironment of ccRCC tumors, which is very different from the microenvironment of the dopaminergic synaptic cleft, the SLC6A3 protein is functional. Thus, SLC6A3 can be used as a target for diagnosis and therapy of ccRCC using compounds specifically transported by, or binding to, SLC6A3. These compounds could either be linked to cytotoxic moieties, immunomodulatory moieties or radioactive labels for highly specific therapy or imaging of ccRCC.
Thus, it is one aspect of the invention to provide agents comprising or consisting of SLC6A3 ligand linked to a radioactive label, a cytotoxic moiety or an immunomodulatory moiety for use in a method for diagnosis or treatment of clear cell renal cell carcinoma (ccRCC) in an individual.
It is also an aspect of the invention to provide methods for diagnosing ccRCC in an individual, said method comprising the steps of
a) administering an agent comprising or consisting of a SLC6A3 ligand linked to a radioactive label to an individual
b) detecting the localised presence of said agent outside the central nervous system in said individual
   wherein the localised presence of said agent outside the central nervous system in the individual is indicative of the presence of ccRCC or metastasized ccRCC in said individual.

It is also an aspect of the invention to provide methods of treatment of ccRCC in an individual in need thereof, said method comprising administering an agent comprising or consisting of a SLC6A3 ligand linked to a radioactive label, a cytotoxic moiety or an immunomodulatory moiety to said individual in a pharmaceutically effective amount.

### DESCRIPTION OF DRAWINGS

FIG. 1 shows expression of SLC6A3 and controls. Panel A shows a boxplot summarizing the expression of the prototypical hypoxia-induced gene CAIX across The Cancer Genome Atlas (TCGA) data set of 25 different tumor types (log 2 expression, N=normal, T=tumor). Panel B shows a boxplot summarizing the expression of SLC6A3 across 25 different tumor types represented in the TCGA data sets (log 2 expression, N=normal, T=tumor).

1. Adrenocortical carcinoma (ACC), 2. Urothelial bladder cancer (BLCA), 3. Breast cancer (BRCA), 4. Cervical cancer (CESC), 5. Colon adenocarcinoma (COAD), 6. Glioblastoma multiforme (GBM), 7. Head and neck squamous cell carcinoma (HNSC), 8. Chromophobe renal cell carcinoma (KICH), 9. Clear cell kidney carcinoma (KIRC), 10. Papillary kidney carcinoma (KIRP), 11. Lower grade glioma (LGG), 12. Liver hepatocellular carcinoma (LIHC), 13. Lung adenocarcinoma (LUAD), 14. Lung squamous cell carcinoma (LUSC), 15. Mesothelioma (MESO), 16. Ovarian serous cystadenocarcinoma (OV), 17. Pancreatic ductal adenocarcinoma (PAAD), 18. Pheochromocytoma and Paraganglioma (PCPG), 19 Prostate adenocarcinoma (PRAD), 20. Rectal adenocarcinoma (READ), 21. Sarcoma (SARC), 22. Cutaneous melanoma (SKCM), 23. Papillary thyroid carcinoma (THCA), 24. Uterine corpus endometrial carcinoma (UCEC) and 25. Uterine carcinosarcoma (UCS).

Panel C shows expression of SLC6A3 across normal tissue samples, showing that the gene is expressed in specific regions of the CNS, but not in normal kidney. Boxplot (log 2 expression) of SLC6A3 gene expression in 353 post mortem samples collected from 20 anatomically distinct sites of the human central nervous system (CNS) and 45 non-CNS tissues (GSE3526; Roth et al.). Sample 1 and 2, substantia nigra and ventral segment area, respectively. Sample 33 and 59, kidney medulla and kidney cortex, respectively. Panel D shows a boxplot (linear expression) of SLC6A3 expression in 53 normal tissue types Lane 20, brain substantia nigra, lane 34 normal kidney. Data derived from GTEx portal release V6.

FIG. 2 shows distribution of gene specific reads through the 15 coding exons of SLC6A3 (5′ to 3′) based on analyses of TOGA cohort of 531 ccRCC (A), 127 normal kidney samples (B), 66 chromophobe RCC (C) and 237 papillary RCC (D). The median number of reads of each exon is equally distributed across the gene indicating a normal initiation and read-though of the gene in the ccRCC cohort.

FIG. 3 shows the uptake of [³H]-dopamine over time (in minutes) by primary renal cell carcinoma cells (1) and by matched primary normal kidney cells (2) from the same patient. Uptake was assessed by radioactivity related to pmol of [³H]dopamine per assay well.

FIG. 4: SPECT/CT images of a mouse injected with 5.36 MBq (¹²³I-Ioflupan). Image 1 h and 35 mins after injection showing uptake of (¹²³I-Ioflupan) in the left kidney injected with KMRC-3 cells, while no signal could be detected in the contralateral kidney.

FIG. 5 shows expression analysis of SLC6A3 in 16 matched primary ccRCC tumors (ccrcc) and metastasis (ccrcc.met). SLC6A3 is expressed in both primary ccRCC (1) and in 35 metastasis (2). From GSE23629 (López-Lago M A, 2010)

FIG. 6 shows that SLC6A3 expression is present in primary ccRCC, KMRC3 and SNU-349 but lost in other conventional cell lines.

Panel A shows relative mRNA expression of SLC6A3 in ccRCC-cell lines and breast cancer cell line MCF7 as control. 1. SNU-349, 2. KMRC3, 3. SKRC10, 4. RCC4, 5. RCC4 +VHL 6. RCB1963, 7. SKRC7, 8. SKRC17, 9. SKRC21, 10. 786-O, 11. WT7, 12. MCF7. Panel B shows relative mRNA expression of SLC6A3 in short term culture of primary renal cells (3-6) from matched normal (1) and tumor (2) and display upregulated SLC6A3 in tumor samples p=0.025 (paired 2-tailed student t-test). Data presented as mean±SEM and mRNA levels were related to housekeeping genes SDHA, UBC and YWHAZ.

FIG. 7 shows that primary ccRCC cells actively take up dopamine.

Panel A shows [³H]dopamine uptake in short term cultured primary renal cells from a matched tumor (1) and normal (2) sample exposed to [³H]dopamine in the absence (3) or presence of competitive unlabeled dopamine 2 μM (4) or 20 μM (5). Uptake was assessed by radioactivity related to pmol of [³H]dopamine per assay well. Panel B shows [³H]dopamine uptake in four short term cultured primary ccRCC cells samples (4-7) treated as in as (A). Ctrl (1), 2 μM competitive unlabeled dopamine (2) or 20 μM competitive unlabeled dopamine (3). Data presented as mean±SD. Statistical significance was calculated using 2-tailed students t-test * p<0.05; ** p<0.01; *** p<0.001.

FIG. 8 show that dopamine uptake in ccRCC cell lines can be diminished using specific SLC6A3 inhibitors.

Panel A shows [³H]dopamine uptake of SKRC10 cells in the absence (1) or presence (2) of 30 nM GBR12935 for 5 min before abortion of uptake by ice cold wash buffer. Panel B shows [³H]dopamine uptake of KMRC3 cells in the absence (1) or presence of GBR12909 at 10 nM (2), 30 nM (3) or 90 nM (4) for 5 min before abortion of uptake by ice cold wash buffer. Panel C shows [³H]dopamine uptake of SNU-349 cells in the absence (1) or presence of GBR12909 at 10 nM (2), 30 nM (3) or 90 nM (4) for 5 min before abortion of uptake by ice cold wash buffer. For (A) Uptake was assessed by radioactivity related to pmol of [³H]dopamine per assay well and for (B-C) to pmol of [³H]dopamine per ug protein. Data presented as mean±SEM. Statistical significance was calculated using 2-tailed paired students t-test * p<0.05; ** p<0.01; *** p<0.001.

### DETAILED DESCRIPTION OF THE INVENTION

### Definitions

The term “alkoxy” as used herein refers to O-alkyl. Preferably alkoxy is C₁₋₆-alkoxy, such as C₁₋₃-alkoxy.

The term “alkyl” as used herein refers to a saturated, straight or branched hydrocarbon chain. The hydrocarbon chain preferably contains of from one to six carbon atoms (C₁₋₆-alkyl), including methyl, ethyl, propyl, isopropyl, butyl, isobutyl, secondary butyl, tertiary butyl, pentyl, isopentyl, neopentyl, tertiary pentyl, hexyl and isohexyl.

The term “alkenyl” as used herein refers to a saturated, straight or branched hydrocarbon chain containing at least one double bond. The hydrocarbon chain preferably contains of from two to six carbon atoms (C₂₋₆-alkenyl). Alkenyl may preferably be any of the alkyls described above (except methyl) containing one or more double bonds.

The term “amphetamine analogue” as used herein refers to compounds which specifically binds to SLC6A3 and which are transported across the cellular membrane by SLC6A3, and which do not bind to the dopamine receptor with any significant affinity. Thus, an amphetamine analogue preferably binds SLC6A3 with at least 3×, such as at least 5× higher affinity than binding to dopamine receptor. Thus, amphetamine analogues are compounds having a function vis-à-vis SLC6A3, which is similar to amphetamine.

The term “aryl” as used herein refers to a substituent derived from an arene by removal of one H from a C in the ring. Examples of useful aryls to be used with the present invention comprise phenyl, napthyl, anthracenyl, phenanthrenyl, and pyrenyl.

The term halogen as used herein refers to a substituent selected from the group consisting of —F, —Cl, —Br and —I.

The term “heteroaryl” as used herein refers to a substituent derived from an heteroarene by removal of one —H from an atom in the ring structure of said heteroarene. Heteroarenes are mono- or polycyclic aromatic compounds comprising one or more heteroatoms in the ring structure. Said heteroatoms are preferably selected from the group consisting of S, N and 0.

The term “ccRCC” as used herein refers to clear cell renal cell carcinoma. ccRCC is typically characterized by malignant epithelial cells with clear cytoplasm. ccRCC display an almost universal loss of the tumor suppressor gene von Hippel Lindau (VHL), and ccRCC is typically characterized by a strong induction of angiogenesis and at the same time display a unique metabolic profile based on glycolysis.

The term “cocaine analogues” as used herein refers to compounds which specifically binds SLC6A3, but which are not transported across the cellular membrane by SLC6A3, and which do not bind to the dopamine receptor with any significant affinity. Thus, a cocaine analogue preferably binds SLC6A3 with at least 3×, such as at least 5× higher affinity than binding to dopamine receptor. Thus, cocaine analogues are compounds having a function vis-à-vis SLC6A3, which is similar to cocaine.

The term “SLC6A3 ligand” as used herein refers to compounds, which specifically binds to SLC6A3 and/or which specifically are transported across the cellular membrane by SLC6A3. For example the SLC6A3 ligand may be a compound, which is transported from the surroundings and across the cellular membrane into the cytoplasm by SLC6A3. It is preferred that the SLC6A3 ligand has a high affinity for SLC6A3 with a Ki of at the most 100 nM. Furthermore it is preferred that the SLC6A3 ligand has an affinity for SLC6A3, which is at least 10 times higher than the affinity for SERT. It is also preferred that the SLC6A3 ligand has higher affinity for SLC6A3 than for the dopamine receptor.

The term “substituted with X” as used herein in relation to organic compounds refers to one hydrogen molecule of said organic compound being substituted with X.

### Agent

The present invention relates to an agent comprising or consisting of an SLC6A3 ligand linked to a radioactive label, a cytotoxic moiety or an immunomodulatory moiety.

Said agent may in one embodiment consist of an SLC6A3 ligand linked to a radioactive label, a cytotoxic moiety or an immunomodulatory moiety.

The SLC6A3 ligand may be any of the SLC6A3 ligands described herein below in the section “SLC6A3 ligand”, for example any of the compounds listed in Table 1. The radioactive label may be any of the labels mentioned herein below in the section “Radioactive label”. The cytotoxic moiety or the immunomodulatory modity may be any of the cytotoxic or immunomodulatory moieties described in the section “Cytotoxic or immunomodulatory moiety” herein below.

The SLC6A3 ligand may be linked to said radioactive label, cytotoxic moiety or immunomodulatory moiety by any suitable means, but in one embodiment of the invention, the SLC6A3 ligand is covalently linked to said radioactive label, cytotoxic moiety or immunomodulatory moiety. The SLC6A3 ligand may be directly linked to the radioactive label, cytotoxic moiety or immunomodulatory moiety via one covalent bond, but it is also comprised within the invention that the SLC6A3 ligand is linked to the radioactive label, cytotoxic moiety or immunomodulatory via a linker.

Typically, when the SLC6A3 ligand is linked to a radioactive label, then this is achieved by substituting at least one atom of said SLC6A3 ligand with a radioactive isotope of said atom. Alternatively, the SLC6A3 ligand may be linked to a radioactive isotope via a covalent bond.

In embodiments of the invention where the SLC6A3 ligand is linked to a cytotoxic moiety or immunomodulatory, this may frequently be via a short linker.

The agent according to the present invention may be for use in treatment of ccRCC as described below in the section “Therapeutic method”. The agent according to the invention may also be for use in a diagnostic method for diagnosis of ccRCC as for example described below in the section “Diagnostic method”.

### Diagnostic Method

In one embodiment the present invention relates to an agent for use in a method of diagnosing ccRCC. In such embodiments the agent preferably comprises a SLC6A3 ligand linked to a radioactive label.

The methods of diagnosis typically comprise the steps of:
a) administering an agent comprising or consisting of a SLC6A3 ligand linked to a radioactive label to an individual
b) detecting the localised presence of said agent outside the central nervous system in said individual.

The SLC6A3 ligand may be linked to a radioactive label, for example the SLC6A3 ligand may be linked to a radioactive label by a covalent bond. It is also comprised within the invention that the SLC6A3 ligand linked to a radioactive label, is a SLC6A3 ligand, wherein at least one atom has been exchanged for a radioactive isotope, such as any of the radioactive labels described herein below in the section “Radioactive label”.

The SLC6A3 ligand may be any of the SLC6A3 ligands described herein below in the section “SLC6A3 ligand”. The SLC6A3 ligand for use in diagnostic methods may in some embodiments be an SLC6A3 ligand, which is capable of specifically binding SLC6A3, but which are not transported across the cellular membrane by SLC6A3. Thus, the SLC6A3 ligand for use in the diagnostic methods of the invention may for example be a cocaine analogue.

The agent for use in the diagnostic methods of the invention may for example comprise or consist of any of the compounds mentioned in Table 1 below.

The SLC6A3 ligand is administered by any useful means. Frequently, the agent comprising the SLC6A3 ligand is prepared for parenteral administration.

Parenteral administration is any administration route not being the oral/enteral route whereby the medicament avoids first-pass degradation in the liver. Accordingly, parenteral administration includes any injections and infusions, for example bolus injection or continuous infusion, such as intravenous administration, intramuscular administration, subcutaneous administration. Furthermore, parenteral administration includes inhalations and topical administration.

In one embodiment of the agent comprising the SLC6A3 ligand is prepared for intravenous administration. Said intravenous administration may be administration by injection of one or more doses. Some SLC6A3 ligands linked to a radioactive label, such as Ioflupane (¹²³I) are commercially available in dosage units prepared for intravenous administration.

Intravenous administration may be performed in any suitable way, for example via a peripheral intravenous cannula.

When using a SLC6A3 ligand linked to radioactive iodine, it may be preferable to block thyroid uptake of radioactive iodine. This may be achieved by administration of iodine to the individual, for example by administration of an iodide salt, e.g. potassium iodide. Said iodide salt may be administered by any suitable route, such as by oral administration. Typically, said iodide salt may be administered prior to and/or subsequent to administration of the SLC6A3 ligand linked to radioactive iodine, for example in the range of 1 to 3 hours prior to and/or 12 to 36 hours subsequent to administration of the SLC6A3 ligand linked to radioactive iodine.

After administration of the agent comprising the SLC6A3 ligand to the individual, the localised presence of the SLC6A3 is detected. Typically, at least some time must pass between administration and detection, because the SLC6A3 needs sufficient time to localise to the tissues expressing SLC6A3 before detection is performed. Thus, detection may for example be performed from 1 to 24 hours, such as from 1 to 12 hours, for example from 2 to 8 hours, such as from 3 to 6 hours post administration of the agent comprising the SLC6A3 ligand.

Detection may be done by any useful method. Some detection methods may be more useful for detection of some radioactive isotopes, whereas others are more useful for detection of other radioisotopes.

In one embodiment the presence of the agent is detected using an imaging technique using gamma rays. This may be the case when the SLC6A3 ligand is linked to a gamma-emitting radioisotope. The imaging technique using gamma rays may for example be conventional nuclear medicine planar imaging using a gamma camera or it may be single-photon emission computed tomography (SPECT). Such a method may for example be useful in embodiments where the SLC6A3 is linked to ¹²³I.

In another embodiment the presence of the agent is detected using an imaging technique detecting pairs of gamma rays emitted indirectly by a positron-emitting radionuclide. An example of such an imaging technique is positron emission tomography (PET). In some embodiments, PET may advantageously be combined with a CT scan. Thus, the presence of the agent may be determined by PET or PET/CT. Such a method may for example be useful in embodiments where the SLC6A3 is linked to a radioactive label, e.g. ¹²⁵I.

As explained above, SLC6A3 is normally expressed primarily in dopaminergic neurons of the central nervous system. Accordingly, a SLC6A3 ligand may localise to the central nervous system, and in particular to the brain. However, SLC6A3 is generally not expressed in other tissues level at any appreciable, and in particularly SLC6A3 is not expressed in normal kidney.

Furthermore, as disclosed herein, ccRCC express SLC6A3 at high levels, both in the primary tumor and in metastases, whereas other malignancies analyzed do not express SLC6A3 at any appreciable level.

Accordingly, if a SLC6A3 ligand localises to a tissue outside the central nervous system, then that is indicative of the individual is suffering from ccRCC. Since primary ccRCC is localised to the kidney, then localised presence of the SLC6A3 ligand outside the central nervous system and outside the kidney is indicative of the metastasized ccRCC.

One method for diagnosis of metastasized ccRCC is administering the agent comprising a SLC6A3 ligand linked to a radioactive label to an individual, and detecting the localised presence of said SLC6A3 ligand in the lymphnodes of said individual. Localised presence of said SLC6A3 ligand in the lymphnodes is indicative of metastasized ccRCC in said individual. The lymphnodes may be any lymphnodes, but frequently, the lymphnodes positioned closest to the kidney may be investigated.

In one embodiment, the method for diagnosis of ccRCC comprises administering the agent comprising a SLC6A3 ligand linked to a radioactive label to an individual and detection of presence of the agent in the kidney of said individual. Presence of said agent in the kidney of the individual is indicative of the presence of ccRCC.

### Therapeutic Method

In one embodiment the present invention relates to an agent for use in a method of treatment of ccRCC. In such embodiments the agent preferably comprises administering an agent comprising or consisting of a SLC6A3 ligand linked to a radioactive label, a cytotoxic moiety or an immunomodulatory moiety to an individual in need thereof in a pharmaceutically effective amount.

The SLC6A3 ligand may be linked to a radioactive label, for example the SLC6A3 ligand may be linked to a radioactive label by a covalent bond. Said radioactive label is preferably a radioactive isotope, which emit sufficient radiation to be cytotoxic or which can be induced to emit sufficient radiation to be cytotoxic. Such a radioactive isotope may also be referred to as a “radioactive label with cytotoxic properties”. For example the radioactive label may be a β-emitter, such as ¹³¹I. Examples of useful radioactive labels are described below in the section “Radioactive label”.

The SLC6A3 ligand for use in methods of treatment of ccRCC may also be linked to a cytotoxic moiety and/or an immunomodulatory moiety, such as any of the moiety described herein below in the section “Cytotoxic or immunomodulatory moiety”.

The SLC6A3 ligand may be any of the SLC6A3 ligands described herein below in the section “SLC6A3 ligand”.

The SLC6A3 ligand for use in method of treatment may in some embodiments be an SLC6A3 ligand, which is capable of specifically binding SLC6A3, but which are not transported across the cellular membrane by SLC6A3. Thus, the SLC6A3 ligand for use in the diagnostic methods of the invention may for example be a cocaine analogue. This may in particular be the case in embodiments wherein the SLC6A3 ligand is linked to a radioactive label.

The SLC6A3 ligand for use in method of treatment may in some embodiments be an SLC6A3 ligand, which is capable of specifically binding SLC6A3, and which is transported across the cellular membrane by SLC6A3. Thus, the SLC6A3 ligand for use in the diagnostic methods of the invention may for example be an amphetamine analogue. This may in particular be the case in embodiments wherein the SLC6A3 ligand is linked to a cytotoxic moiety or an immunomodulatory moiety.

The SLC6A3 ligand is administered by any useful means. Frequently, the agent comprising the SLC6A3 ligand is prepared for parenteral administration. Parenteral administration may be as described above in the section “Diagnostic method”.

In one embodiment of the agent comprising the SLC6A3 ligand for use in treatment of ccRCC is prepared for intravenous administration. Said intravenous administration may be administration by injection of one or more doses. Frequently, several administrations of said agent may be advantageous, such as in the range of 1 to 100 administrations, for example in the range of 1 to 50 administrations.

Intravenous administration may be performed in any suitable way, for example via a peripheral intravenous cannula.

### SLC6A3 Ligand

The SLC6A3 ligand to be used with the present invention may be any compound capable of specifically binding to SLC6A3. SLC6A3 is also known as the dopamine transporter. The sequence of human SLC6A3 is available in the UniProt database under the accession number Q01959-1. The relevant sequence was entered to the database 1 Apr. 1993.

Dopamine is capable of binding to several membrane proteins, including SLC6A3 as well as the dopamine receptor. In one embodiment it is preferred that the SLC6A3 ligand is capable of binding to SLC6A3 with much higher affinity than the dopamine receptor. Thus, the SLC6A3 ligand may bind SLC6A3 with at least 3×, such as at least 5× higher affinity than binding to dopamine receptor.

The SLC6A3 may in some embodiments of the invention be an amphetamine analogue. This may in particular be the case in embodiments of the invention relating to methods for treatment of ccRCC, wherein the SLC6A3 ligand is linked to a cytotoxic moiety or an immunomodulatory moiety.

The SLC6A3 may in some embodiments of the invention be a cocaine analogue. This may in particular be the case in embodiments of the invention relating to methods of diagnosis and/or methods of treatment wherein the SLC6A3 ligand is linked to a radioactive label.

In embodiments of the invention relating to methods of treatment it is furthermore preferred that the SLC6A3 ligand is not capable of passing the blood-brain barrier. A non-limiting example of an SLC6A3 ligand, which does not pass the blood-brain barrier is FMIP (see e.g. De Bruyne et al., 2009 and 2010)

It is generally preferred that the SLC6A3 ligand has a high affinity for SLC6A3. Thus, it is preferred that the SLC6A3 ligand has an affinity for SLC6A3 with a Ki of at the most 100 nM. The Ki is the inhibitory constant and it may for example be determined as described in Boos et al., 2006 and the references described therein, notably as described in Greiner et al., 2003. The Ki may also be determined as described in Hong et al., 2016 and reference described therein.

It may also be preferred that the SLC6A3 ligand has a high affinity for SLC6A3 with a Kd of at the most 10,000 nM, preferably of at the most 1000 nM. The Kd is the dissociation constant and it may be determined as for example described in Huot et al., 2015, and in the references described therein.

It is furthermore, preferred that the SLC6A3 ligand has high specificity for SLC6A3. Thus, as described above it is preferred that the SLC6A3 ligand bind to SLC6A3 with higher affinity as to dopamine receptor. Thus, it is preferred that the Kd of the SLC6A3 ligand in respect of the dopamine receptor is at least 3 times, such as at least 5 times higher than the Kd of the SLC6A3 ligand in respect of SLC6A3.

It is also preferred that the SLC6A3 ligand bind to SLC6A3 with much higher affinity as to the serotonin receptor (SERT). Thus, it is preferred at the Ki of the SLC6A3 ligand in respect of SERT is at least 5 times, preferably at least 10 times higher than the Ki of the SLC6A3 ligand in respect of SLC6A3. It may also be preferred at the Kd of the SLC6A3 ligand in respect of SERT is at least 5 times, preferably at least 10 times higher than the Kd of the SLC6A3 ligand in respect of SLC6A3.

In one embodiment of the invention the SLC6A3 ligand is a tropane derivative or a nortropane derivative. Tropane is a compound of the formula:

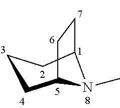

A derivative of tropane according to the invention is a compound wherein one or more hydrogens of tropane has been substituted with another substituent.

Nortropane is a compound of the formula:

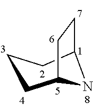

A derivative of nortropane according to the invention is a compound wherein one or more hydrogens of nortropane has been substituted with another substituent.

For example, the SLC6A3 ligand may be tropane or nortropane substituted at the 3 position with phenyl, wherein said tropane, nortropane and phenyl optionally may be further substituted with one or more substituents.

In one embodiment of the invention the SLC6A3 ligand may be a compound of formula I:

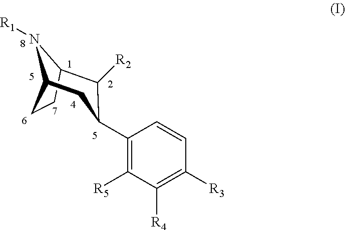

wherein
R₁ is H, C₁₋₆-alkyl, C₂₋₆-alkenyl, phenyl or benzyl, wherein said C₁₋₆-alkyl, C₂₋₆-alkenyl phenyl or benzyl optionally may be substituted with one or more halogen or NH₂;
R₂ is CO₂—R₆, C═N—O—R₆ or heteroaryl-R₆, wherein R₆ is C₁₋₆-alkyl, C₂₋₆-alkenyl, phenyl or benzyl, wherein said C₁₋₆-alkyl, C₂₋₆-alkenyl phenyl or benzyl optionally may be substituted with one or more halogen;
R₃ is C₁₋₃-alkyl, halogen or heteroaryl, wherein said C₁₋₃-alkyl optionally may be substituted with halogen; and
R₄ and R₅ individually are H, C₁₋₃-alkyl or halogen, wherein said C₁₋₃-alkyl optionally may be substituted with halogen.

In one embodiment, R₁ may for example be C₁₋₆-alkyl or C₂₋₆-alkenyl, wherein said C₁₋₆-alkyl or C₂₋₆-alkenyl optionally may be substituted with one or more halogen.

In one embodiment R₁ may be, C₁₋₆-alkyl, C₂₋₆-alkenyl, phenyl or benzyl, wherein said C₁₋₆-alkyl, C₂₋₆-alkenyl phenyl or benzyl optionally may be substituted with one or more halogen. In another embodiment R₁ may be C₁₋₄-alkyl or C₂₋₄-alkenyl, wherein said C₁₋₄-alkyl or C₂₋₄-alkenyl optionally may be substituted with halogen.

Thus, for example R₁ may be C₁₋₄-alkyl or C₂₋₄-alkenyl, wherein said C₁₋₄-alkyl or C₂₋₄-alkenyl is substituted with at least one halogen. Said halogen may in particular be selected from the group consisting of —F and —I.

In one embodiment, R₂ may be CO₂—R₆, wherein R₆ may be C₁₋₆-alkyl or C₂₋₆-alkenyl, wherein said C₁₋₆-alkyl or C₂₋₆-alkenyl optionally may be substituted with one or more halogen.

In one embodiment R₂ may be CO₂—R₆, wherein R₆ is C₁₋₄-alkyl, wherein said C₁₋₄-alkyl optionally may be substituted with halogen.

Thus, for example R₂ may be CO₂—R₆, wherein R₆ may be C₁₋₄-alkyl, wherein said C₁₋₄-alkyl is substituted with at least one halogen. Said halogen may in particular be selected from the group consisting of —F and —I.

In one embodiment R₂ is CO₂—R₆, wherein R₆ is unsubstituted C₁₋₃-alkyl, such as methyl.

In one embodiment, R₃ is C₁₋₃-alkyl or halogen, wherein said C₁₋₃-alkyl optionally may be substituted with halogen.

In one embodiment R₃ is methyl or halogen. Said halogen may be any halogen, i.e. said halogen may be selected from the group consisting of —F, —Br, —Cl and —I.

In one embodiment R₄ and R₅ individually may be —H, C₁₋₃-alkyl or halogen, wherein said C₁₋₃-alkyl optionally may be substituted with halogen.

In one embodiment both R₄ and R₅ may be —H,

In one embodiment the SLC6A3 ligand is a compound of formula I, wherein

R₁ is C₁₋₆-alkyl or C₂₋₆-alkenyl, wherein said C₁₋₆-alkyl or C₂₋₆-alkenyl optionally may be substituted with one or more halogen;
R₂ is CO₂—R₆, wherein R₆ is C₁₋₆-alkyl or C₂₋₆-alkenyl, wherein said C₁₋₆-alkyl or C₂₋₆-alkenyl optionally may be substituted with one or more halogen;
R₃ is C₁₋₃-alkyl or halogen, wherein said C₁₋₃-alkyl optionally may be substituted with halogen; and
R₄ and R₅ individually are H, C₁₋₃-alkyl or halogen, wherein said C₁₋₃-alkyl optionally may be substituted with halogen.

In one embodiment the SLC6A3 ligand is a compound of formula I, wherein

R₁ is C₁₋₄-alkyl or C₂₋₄-alkenyl, wherein said C₁₋₄-alkyl or C₂₋₄-alkenyl optionally may be substituted with halogen;
R₂ is CO₂—R₆, wherein R₆ is C₁₋₄-alkyl, wherein said C₁₋₄-alkyl optionally may be substituted with halogen;
R₃ is C₁₋₃-alkyl or halogen, wherein said C₁₋₃-alkyl optionally may be substituted with halogen; and
R₄ and R₅ individually are H, C₁₋₃-alkyl or halogen, wherein said C₁₋₃-alkyl optionally may be substituted with halogen.

In general it is preferred that the SLC6A3 ligands of formula I described herein above comprises at least one halogen. Said halogen may be any halogen, i.e. a halogen selected from the group consisting of —F, —Br, —Cl and —I. Preferably the SLC6A3 ligands of formula I described herein above comprises at least one halogen selected from the group consisting of —F and —I. In addition to said —F and —I the SLC6A3 ligand may comprise one or more additional halogens.

Thus, in the SLC6A3 ligand of formula I, then at least one of R₁, R₂, R₃, R₄ or R₅ may comprises halogen.

It is preferred that the SLC6A3 ligands of formula I described herein above comprises at least one halogen, wherein said halogen is a radioactive isotope of said halogen. In addition to said radioactive isotope of a halogen, the SLC6A3 ligand may comprise one or more additional halogens. Thus, in the SLC6A3 ligand of formula I, then at least one of R₁, R₂, R₃, R₄ or R₅ may comprise a radioactive isotope of a halogen. The radioactive isotope may be any of the radioactive isotopes mentioned herein below in the section “Radioactive label”.

In another embodiment of the invention, the SLC6A3 ligand may be a compound of formula (II):

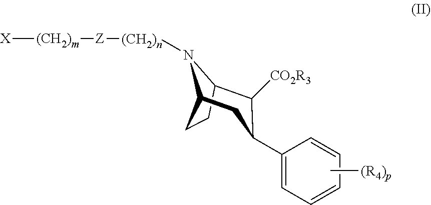

wherein:
X is —Cl, —Br, —I or a group OSO₂R_{c}; —Z is RₐC═CR_{b} or ethynyl; Rₐ and R_{b} are each independently H, C₁₋₆ alkyl, C₃₋₁₀ aryl, wherein said alkyl or aryl groups are optionally substituted by one to three R_{d} groups;
R₃ in relation to compounds of formula II is H or C₁₋₆ alkyl, wherein said alkyl group is optionally substituted by one to three R_{d} groups;
p is 1, 2 or 3;
R₄ in relation to compounds of formula II is/are independently H, Cl, Br, I, F, or C₁₋₆ alkyl, wherein said alkyl group is/are optionally substituted by one to three R_{d} groups;
R_{c} is a C₁₋₆ alkyl, preferably C₁₋₄ alkyl, said alkyl being optionally mono-, poly- or perhalogenated, a C₃₋₈ cycloalkyl or a C₆₋₁₂ aryl, wherein said alkyl, cycloalkyl and aryl are optionally substituted by one to three C₁₋₄ alkyl groups or F, Cl, Br, NO₂, or CN;
R_{d} is OH, F, Cl, Br, phenyl or methyl,
m and n are the same or different and are an integer ranging from 1 to 8. Preferably, m+n<10. More preferably, m is 1, 2, 3 or 4, and n is 1, 2, 3 or 4.
   X may be a OSO₂R_{c} group, for example selected from OSO₂—C₆H₄—CH₃, OSO₂—C₆H₄—Br, OSO₂—C₆H₄—NO₂, OSO₂—CH₃, OSO₂—CF₃, OSO₂—C₄F₉ or OSO₂—CH₂—CF₃. Of these, OSO₂—C₆H₄—CH₃, OSO₂—CF₃, OSO₂—CH₃, commonly designates as tolyloxy (OTs), mesyloxy (OMs) and trifyloxy (OTf) respectively.
   X may be Cl, Br, I, for example X may be Cl. Z may be RₐC═CR_{b} or ethynyl. For example Z may be RₐC═CR_{b}, such as trans RₐC═CR_{b}.
   Rₐ and R_{b} may for example independently selected from H and C₁₋₄ alkyl, notably methyl or ethyl. For example, Rₐ and R_{b} may be H.

In one embodiment m may be 1. In one embodiment n may be 1.

In one embodiment the SLC6A3 ligand is an 3β-aryl tropane, such as any of the 3β-aryl tropane described by Hong et al., 2016. For example, the SLC6A3 ligand may be a compound of formula IIIa or IIIb:

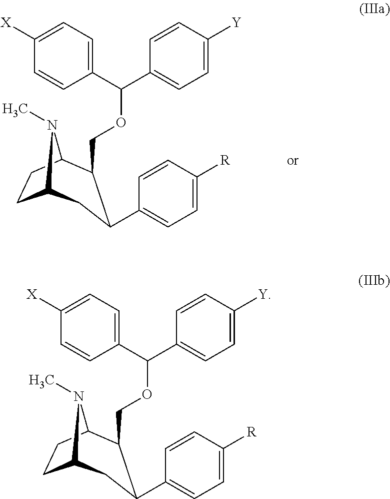

In relation to compounds of formula IIIa and IIIb X, Y and R may be any useful substituent, for example a substituent selected from the group consisting of H and halogen, e.g. —Cl—.

In particular, the SLC6A3 ligand may be selected from the group consisting of compounds LX-10, LX-11, LX-13, LX-12, LX-16, LX-15, LX-19, LX-20, LX-21, LX-22, LX-23 and LX-24 (described e.g. in Hong et al., 2016). In particular, the SLC6A3 ligand may be 2β-Ph2COCH2-3β-4-Cl-Ph.

In one embodiment of the invention the SLC6A3 ligand is a selective DAT inhibitor, for example any of the DAT inhibitors described by Huot et al. 2015. Thus the SLC6A3 ligand may be selected from the group consisting of:

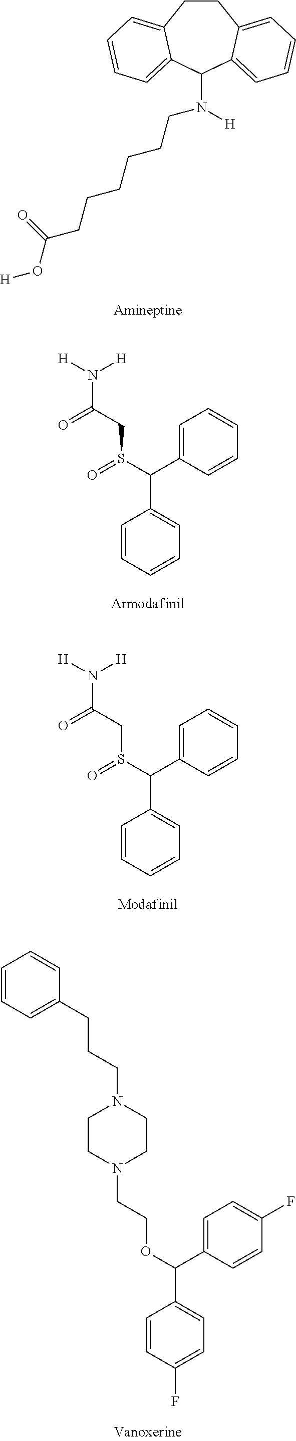 [SMILES: O=C(O)CCCCCCNC1c2ccccc2CCc2ccccc21 ; NC(=O)C[S@](=O)C(c1ccccc1)c1ccccc1 ; NC(=O)CS(=O)C(c1ccccc1)c1ccccc1 ; Fc1ccc(C(OCCN2CCN(CCCc3ccccc3)CC2)c2ccc(F)cc2)cc1]

The SLC6A3 ligand may also be selected from the group consisting of:

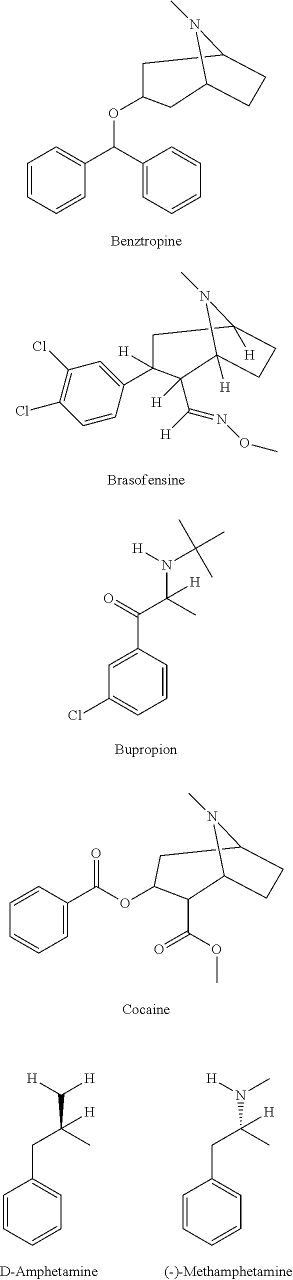

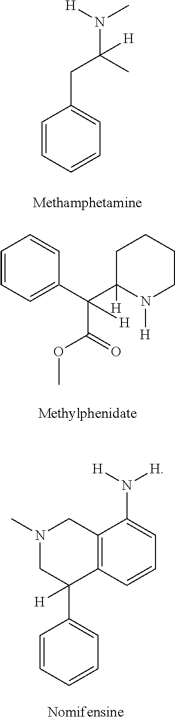

In one embodiment the SLC6A3 ligand may be any of the N-benzyl piperidines of the GBR series described by Boos et al., 2006. Thus, the SLC6A3 ligand may be a compound of the formula IV:

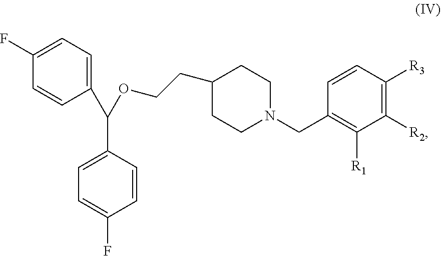

wherein
R₁, R₂ and R₃ individually may be selected from the group consisting of —H, halogen, C₁₋₃-alkyl, C₁₋₃-alkyl substituted with one or more halogen, —CN, and NO₂. Frequently, at least two of R₁, R₂ and R₃ are H, whereas the third is selected from the group consisting of halogen, C₁₋₃-alkyl, C₁₋₃-alkyl substituted with one or more halogen, —CN, and NO₂.

In particular, the SLC6A3 ligand may be any of the compounds 2 to 35 described in Table 1 of Boos et al., 2006.

In one embodiment, at least one of R₁, R₂ and R₃ may comprise or consist of a radioactive isotope of a halogen, e.g. any of the radioactive isotopes of iodine described herein.

In one embodiment the SLC6A3 ligand is a compound of formula IV, wherein R₁ and R₂ are —H and R₃ is —I. This compound is also known as FMIP. In particular the SLC6A3 ligand linked to a radioactive label may be the FMIP linked to a radioactive label, e.g. ¹²³I:

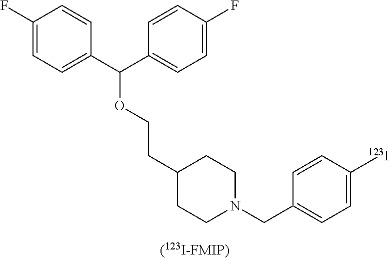 [SMILES: Fc1ccc(C(OCCC2CCN(Cc3ccc([123I])cc3)CC2)c2ccc(F)cc2)cc1]

In one embodiment the SLC6A3 ligand is a compound of formula V:

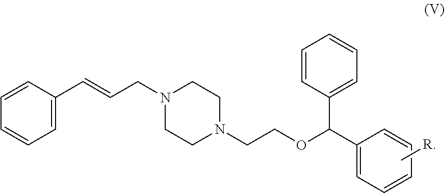

R in relation to formula V may be any useful substituent, for example R may be selected from the group consisting of —H, halogen, —NH2, —NCS, NO₂, and maleimide.

In one embodiment of the invention the SLC6A3 ligand is a GBR-structure. For example the SLC6A3 ligand may be a compound of formula VI:

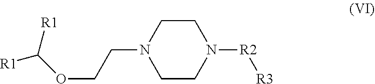

wherein
R₁ is aryl optionally substituted with one or more -halogen, —NH₂, —NCS, —NO₂, or Maleimid-1-yl;
R₂ is C₁₋₃-alkyl, C₁₋₃ alkenyl, —CH₂-cyclopropyl, or —H₂C—C≡C—; and
R₃ is —H, aryl, heteroaryl, bicyclic aromatic ring or heterocyclic ringsystem, which can be partially or fully saturated, wherein any of the aforementioned optionally may be substituted with halogen, —OH, alkoxy or oxy.

In one embodiment R₁ in respect of the compound of formula VI is phenyl optionally substituted with one or more -halogen, —NH₂, —NCS, —NO₂, or Maleimid-1-yl.

In one embodiment R₃ in respect of the compound of formula VI is phenyl.

In one embodiment, the SLC6A3 ligand is a benztropine analogue. For example the SLC6A3 ligand may be a compound of formula VII:

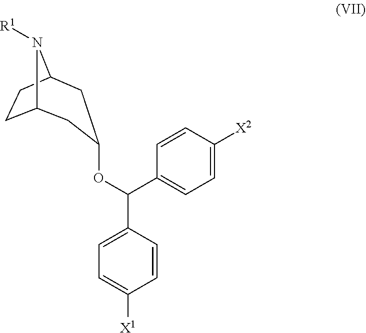

wherein
R₁ is H or L-B; wherein
L is C₁₋₆-alkyl, or —CH₂CH₂—Y—CH₂CH₂—, wherein
   Y is —NH— or —O—; and
B is —OH, halogen, aryl, heterocyclyl, —H, —OR₃, or NHR₂, wherein
   R₂ is —CO-aryl or —CO-heteroaryl; and
      X₁ and X₂ independently are —H, C₁₋₆-alkyl, halogen, —OH, —O—C₁₋₆-alkyl, —NH₂, —NR₃R₄,
      wherein
R₃ and R₄ independently may be —H, C₁₋₆-alkyl, —CN or NO₂.

In one embodiment, the SLC6A3 ligand is a Sulfinacetamide analogue. For example the SLC6A3 ligand may be a compound of formula VIII:

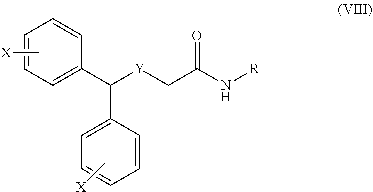

wherein

### Y is —S— or SO;

X is independently —H, C₁₋₆-alkyl, halogen or CN; and
R is C₁₋₆ alkyl or cycloalkyl, optionally substituted with aryl.

The compounds of formula VIII may comprise multiple substituents X, which may be the same or different.

In one embodiment, the SLC6A3 ligand is a Quinazoline analogue. For example the SLC6A3 ligand may be a compound of formula IX:

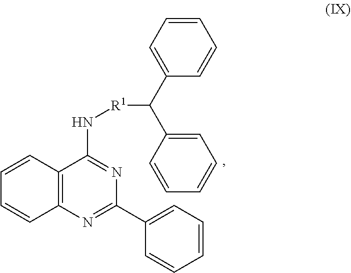

wherein
R₁ may be C₁₋₃ alkyl.

In one embodiment the SLC6A3 ligand is 1-piperazinepropanamine, 4-[2-[4-azido-3-(iodo-)phenyl]ethyl]-N,N-bis(4-fluorophenyl)-9(Cl). In particular the SLC6A3 ligand linked to a radioactive label may be aforementioned linked to a radioactive label, e.g. ¹²⁵I:

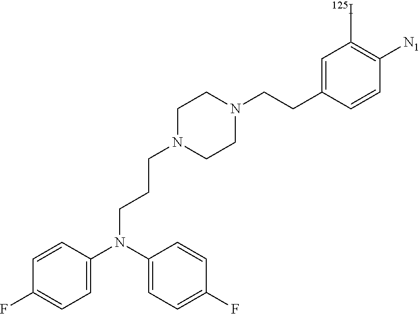

In one embodiment, the SLC6A3 ligand may be one of the following compounds:

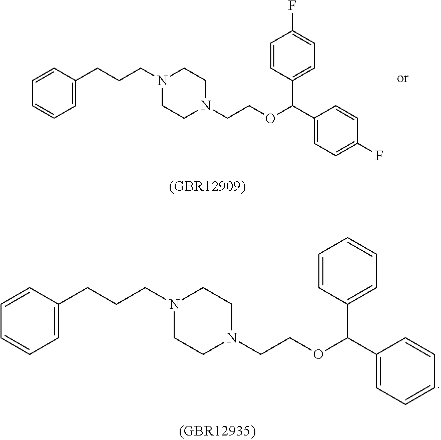 [SMILES: Fc1ccc(C(OCCN2CCN(CCCc3ccccc3)CC2)c2ccc(F)cc2)cc1 ; c1ccc(CCCN2CCN(CCOC(c3ccccc3)c3ccccc3)CC2)cc1]

The SLC6A3 ligand to be used with the methods of the invention may be any of the compounds described herein above, for example any of the compounds of formula I, II, IIIa, IIIb, IV, V, VI, VII, VIII or IX described herein above, or stereoisomeric forms, mixtures of stereoisomeric forms, solvates and salt forms thereof. Thus, the SLC6A3 ligand may be any of the compounds of formula I, II, IIIa, IIIb, IV, V, VI, VII, VIII or IX described above or a pharmaceutically acceptable salt thereof.

In one embodiment of the invention, the SLC6A3 ligand linked to a radioactive label may be any of the radiotracers targeting the dopamine transporter described in Shen et al., 2012.

In one embodiment of the invention the SLC6A3 ligand linked to a radioactive label may be any of the tropane derivatives described in WO2008/059349.

In one embodiment of the invention SLC6A3 ligand may be linked to a radioactive label, and in such embodiments the SLC6A3 ligand linked to a radioactive label may be selected from the group consisting of β-CFT (WIN 35,428), β-CIT (RTI-55), β-CCT (RTI-31), β-CMT (RTI-32), FECNT, β-CCT-FP (β-FPCT), β-CBT, β-FECT (β-FE-CCT), β-FETT (β-FE-CMT), β-FIPCT (β-FiP-CCT), β-CFT-FE, β-CFT-FP, β-CBT-FE, β-CIT-FP (FP-CIT), β-CMT-FP (FP-CMT), β-CBT-FP (FP-CBT), β-CDCT, β-IP-CIT (RTI-121), NS-2214 (BMS-204756), β-C IT-FE, β-CpFMT, β-CmFMT, β-CoFMT (o-FWIN), β-CPPIT, FCT, E-IACFT (Altropane), MCL301, MCL322, FE@CIT, FBCINT, FE-3FPT and PE2I. This may in particular be the case in embodiments of the invention relating to methods for diagnosis.

In embodiments of the invention relating to methods of treatment, the SLC6A3 ligand may for example be selected from the group consisting of β-CFT (WIN 35,428), β-CIT (RTI-55), β-CCT (RTI-31), β-CMT (RTI-32), FECNT, β-CCT-FP (β-FPCT), β-CBT, β-FECT (β-FE-CCT), β-FETT (β-FE-CMT), β-FIPCT (β-FiP-CCT), β-CFT-FE, β-CFT-FP, β-CBT-FE, β-CIT-FP (FP-CIT), β-CMT-FP (FP-CMT), β-CBT-FP (FP-CBT), β-CDCT, β-IP-CIT (RTI-121), NS-2214 (BMS-204756), β-C IT-FE, β-CpFMT, β-CmFMT, β-CoFMT (o-FWIN), β-CPPIT, FCT, E-IACFT (Altropane), MCL301, MCL322, FE@CIT, FBCINT, FE-3FPT and PE2I, wherein
i) the radioactive label of said compounds has been exchanged for a radioactive label with cytotoxic properties (see section “Radioactive label” below); and/or
ii) the radioactive label of said compounds has been removed or exchanged for a non-radioactive counterpart, and the compound has been covalently linked to a radioactive label with cytotoxic properties (see section “Radioactive label” below); and/or
iii) the radioactive label of said compounds has been removed or exchanged for a non-radioactive counterpart, and the compound has been covalently linked to a cytotoxic moiety and/or an immunomodulatory moiety and/or
iv) the compounds are linked to a cytotoxic moiety and/or a immunomodulatory moiety.

In one embodiment of the invention the SLC6A3 ligand linked to a radioactive label may be any of the compounds shown herein below in Table 1 or it may be FMIP comprising or linked to a radioactive label, e.g. ¹²³I-FMIP.

TABLE 1

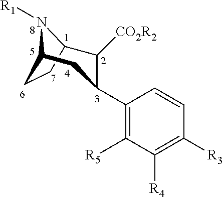

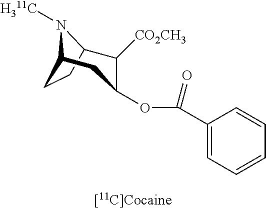

| Code | R₁ | R₂ | R₃ | R₄ | R₅ |
|---|---|---|---|---|---|
| β-CFT (WIN, 35,428) | [¹¹C]CH₃ | [¹¹C]CH₃ | [¹⁸F]F | H | H |
| β-CIT (RIT-55) | [¹¹C]CH₃ | [¹¹C]CH₃ | I | H | H |
| β-CCT (RTI-31) | CH₃ | [¹¹C]CH₃ | Cl | H | H |
| β-CMT (RTI-32) | CH₃ | [¹¹C]CH₃ | CH₃ | H | H |
| FECNT | CH₂CH₂[¹⁸F]F | CH₃ | Cl | H | H |
| β-CCT-FP (β-FPCT) | (CH₂)₂CH₂[¹⁸F]F | CH₃ | Cl | H | H |
| β-CBT | CH₃ | CH₃ | [⁷⁵Br]Br | H | H |
| β-FECT (β-FE-CCT) | CH₃ | CH₂CH₂[¹⁸F]F | Cl | H | H |
| β-FETT (β-FE-CMT) | CH₃ | CH₂CH₂[¹⁸F]F | CH₃ | H | H |
| β-FIPCT (β-FiP-CCT) | CH₃ | CH(CH₃)CH₂[¹⁸F]F | Cl | H | H |
| β-CFT-FE | CH₂CH₂[¹⁸F]F | CH₃ | F | H | H |
| β-CFT-FP | (CH₂)₂CH₂[¹⁸F]F | CH₃ | F | H | H |
| β-CBT-FE | CH₂CH₂F | CH₃ | [⁷⁵Br]Br | H | H |
| β-CIT-FP (FP-CIT) | (CH₂)₂CH₂[¹⁸F]F | CH₃ | I | H | H |
| β-CMT-FP (FP-CMT) | (CH₂)₂CH₂[¹⁸F]F | CH₃ | CH₃ | H | H |
| β-CBT-FP (FP-CBT) | (CH₂)₂CH₂[¹⁸F]F | CH₃ | [⁷⁶Br]Br | H | H |
| β-CDCT | CH₃ | [¹¹C]CH₃ | Cl | Cl | H |
| β-IP-CIT (RTI-121) | [¹¹C]CH₃ | CH(CH₃)₂ | I | H | H |
| NS-2214 (BMS-204756) | [¹¹C]CH₃ | see below ^{(a)} | Cl | Cl | H |
| β-CIT-FE | CH₂CH₂F | [¹¹C]CH₃ | I | H | H |
| β-CpFMT | CH₃ | CH₃ | CH₂[¹⁸F]F | H | H |
| β-CmFMT | CH₃ | CH₃ | H | CH₂[¹⁸F]F | H |
| β-CoFMT (o-FWIN) | CH₃ | CH₃ | H | H | CH₂[¹⁸F]F |
| β-CPPIT | [¹¹C]CH₃ | see below ^{(b)} | Cl | H | H |
| FCT | 4-[¹⁸F]FBn | CH₂CH₃ | Cl | H | H |
| E-IACFT (Altropane) | CH₂CH═CHI | [¹¹C]CH₃ | F | H | H |
| MCL301 | CH₃ | CH₂CH₂[¹⁸F]F | I | H | H |
| MCL322 | CH₃ | CH₂CH₂[¹⁸F]F | Br | H | H |
| FE@CIT | CH₃ | CH₂CH₂[¹⁸F]F | I | H | H |
| FBCINT | CH₂(CH═CH)CH₂[¹⁸F]F | CH₃ | Cl | H | H |
| FE-3FPT | CH₂CH₂[¹⁸F]F | CH₃ | 3′-furyl | H | H |
| PE2I | CH₂CH═CHI | [¹¹C]CH₃ | CH₃ | H | H |

^{(a)} 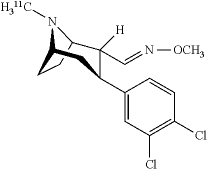
^{(b)}

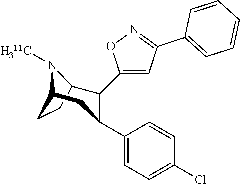

In one embodiment of the invention the SLC6A3 ligand linked to a radioactive label may be selected from the group consisting of ¹²³I-FMIP, [¹¹C]-2β-carbomethoxy-3β-Itropane, 2β-carbomethoxy-3β-(4-fluorophenyl)-N-((E)-3-iodo-prop-2-enyl)tropane, [¹²³I]-(1R)-2-β-Carbomethoxy-3β-(4-iodophenyl)-tropane, [¹²³I] N-ω-fluoropropyl-2β-carbomethoxy-3β-(4-iodophenyl)-nortropane, [^{99m}Tc]technetium [2-[[2-[[[3-(4-chlorophenyl)-8-methyl-8-azabicyclo-[3.2.1]oct-2-yl]-methyl](2-mercaptoethyl)amino]-ethyl]amino]ethanethiolato(3−)-N2,N2′,S2,S2′]oxo-[1R-(exoexo)], [¹²³I]-2β-carbomethoxy-3β-(4-fluorophenyl)-N-(1-iodoprop-1-en-3-yl)-ortropane, and [¹²³I]—N-(3-iodopropen-2-yl)-2-carbomethoxy-3beta-(4-chlorophenyl)-tropane. This may in particular be the case in embodiments of the invention relating to methods for diagnosis.

In another embodiment of the invention the SLC6A3 ligand linked to a radioactive label may be selected from the group consisting of ¹²³I-FMIP, [¹¹C]-2β-carbomethoxy-3β-Itropane, 2β-carbomethoxy-3β-(4-fluorophenyl)-N-((E)-3-iodo-prop-2-enyl)tropane, [¹²³I]-(1R)-2-β-Carbomethoxy-3-β-(4-iodphenyl)-tropane, [¹²³I] N-ω-fluoropropyl-2β-carbomethoxy-3β-(4-iodophenyl)-nortropane, [^{99m}Tc]technetium [2-[[2-[[[3-(4-chlorophenyl)-8-methyl-8-azabicyclo-[3.2.1]oct-2-yl]-methyl](2-mercaptoethyl)amino]-ethyl]amino]ethanethiolato(3−)-N2,N2′,S2,S2′]oxo-[1R-(exoexo)], [¹²³I]-2β-carbomethoxy-3β-(4-fluorophenyl)-N-(1-iodoprop-1-en-3-yl)-ortropane, and [¹²³I]—N-(3-iodopropen-2-yl)-2-carbomethoxy-3beta-(4-chlorophenyl)-tropane. This may in particular be the case in embodiments of the invention relating to methods for diagnosis, wherein at least one atom the aforementioned SLC6A3 ligands has been exchanged for a radioactive isotope, e.g. a radioactive isotope selected from the group consisting of ¹²³I, ¹²⁵I, ¹¹C, ¹⁸F, ⁷⁶Br, ^{99m}Tc, ¹³N, ¹⁵O, ⁶⁸Ga, ⁸⁹Zr and ⁸²Rb.

In embodiments of the invention relating to methods of treatment, the SLC6A3 ligand may for example be selected from the group consisting of ¹²³I-FMIP, [¹¹C]-2β-carbomethoxy-3β-Itropane, 2β-carbomethoxy-3β-(4-fluorophenyl)-N-((E)-3-iodo-prop-2-enyl)tropane, [¹²³I]-(1R)-2-β-Carbomethoxy-3-β-(4-iodophenyl)-tropane, [¹²³I] N-ω-fluoropropyl-2β-carbomethoxy-3β-(4-iodophenyl)-nortropane, [^{99m}Tc]technetium [2-[[2-[[[3-(4-chlorophenyl)-8-methyl-8-azabicyclo-[3.2.1]oct-2-yl]-methyl](2-mercaptoethyl)amino]-ethyl]amino]ethanethiolato(3−)-N2,N2′,S2,S2′]oxo-[1R-(exoexo)], [¹²³I]-2β-carbomethoxy-3β-(4-fluorophenyl)-N-(1-iodoprop-1-en-3-yl)-ortropane, and [¹²³I]—N-(3-iodopropen-2-yl)-2-carbomethoxy-3beta-(4-chlorophenyl)-tropane, wherein
i) the radioactive label of said compounds has been exchanged for a radioactive label with cytotoxic properties (see section “Radioactive label” below); and/or
ii) the radioactive label of said compounds has been removed or exchanged for a non-radioactive counterpart, and the compound has been covalently linked to a radioactive label with cytotoxic properties (see section “Radioactive label” below); and/or
iii) the radioactive label of said compounds has been removed or exchanged for a non-radioactive counterpart, and the compound has been covalently linked to a cytotoxic moiety and/or an immunomodulatory moiety and/or iv) the compounds are linked to a cytotoxic moiety and/or a immunomodulatory moiety.

In one embodiment of the invention the SLC6A3 ligand linked to a radioactive label is [¹²³I] N-ω-fluoropropyl-2β-carbomethoxy-3β-(4-iodophenyl)nortropane.

In one embodiment of the invention the SLC6A3 ligand linked to a radioactive label is ioflupane of the structure:

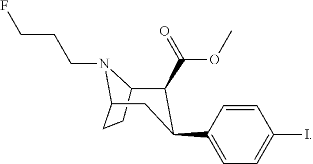 [SMILES: COC(=O)[C@@H]1C2CCC(C[C@@H]1c1ccc(I)cc1)N2CCCF]

The “I” of Ioflupane may be ¹²³I. This may in particular be the case in embodiments of the invention relating to methods for diagnosis.

The “I” of Ioflupane may also be ¹²⁵I. This may in particular be the case in embodiments of the invention relating to methods for diagnosis or treatment.

The “I” of Ioflupane may also be ¹³¹I. This may in particular be the case in embodiments of the invention relating to methods for treatment. Ioflupane (123I) may also be referred to as FP-CIT or as ¹²³I-FP-CIT, and is commercially available under the tradename DaTSCAN.

In one embodiment of the invention the SLC6A3 ligand linked to a radioactive label is ¹²³I-FMIP. The structure of ¹²³I-FMIP is provided above, and ¹²³I-FMIP may in particular be useful in embodiments of the invention relating to methods for diagnosis. The SLC6A3 ligand may also be ¹²⁵I-FMIP being suitable for example for methods for diagnosis or treatment. The SLC6A3 ligand may also be ¹³¹I-FMIP being suitable for example for methods of treatment.

In one embodiment of the invention the SLC6A3 ligand may be an antibody specifically binding to SLC6A3. Since SLC6A3 is a transmembrane protein, it is preferred that such an antibody is capable of specifically binding to the extracellular domain(s) of SLC6A3. The SLC6A3 ligand may in particular be an antibody in embodiments of the invention, wherein the SLC6A3 ligand is linked to a radioactive label.

SLC6A3 may sometimes also be referred to as SCL6A3.

### Radioactive Label

The agent for use in the methods of the invention may be an SLC6A3 ligand linked to a label, preferably a radioactive label. The SLC6A3 ligand linked to a radioactive label may be any of the SLC6A3 ligands described in the section “SLC6A3 ligand” comprising a radioactive label or bound to a radioactive label. Thus, in one embodiment the SLC6A3 ligand linked to a radioactive label is any of the SLC6A3 ligands described in the section “SLC6A3 ligand”, wherein one atom has been exchanged for a radioactive isotope, e.g. any of the radioactive labels described in this section.

The radioactive label may be any useful radioactive label. For example the radioactive label may be selected from the group consisting of ¹²³I, ¹²⁵I, ¹¹C, ¹⁸F, ⁷⁶Br, ^{99m}Tc, ¹³N, ¹⁵O, ⁶⁸Ga, ⁸⁹Zr and ⁸²Rb. This may in particular be the case in embodiments of the invention relating to methods of diagnosis.

Thus, in one embodiment the radioactive label may be ¹²³I.

In general the radioactive label should be sufficiently radioactive to carry out the diagnostic method. The skilled person will be able to determine whether a given radioactive label is sufficiently radioactive.

For example, the activity at the time of administration may be at least 50 MBq, preferably at least 100 MBq, such as at least 111 MBq. For Ioflupane 123I, the activity at the time of administration is typically in the range of 150 to 250 MBq, such as around 185 MBq.

The radioactive label may also be selected from the group consisting to ¹²⁵I, ¹³¹I, ⁸⁹Sr, ¹⁵³Sm and ²²³Ra. This may in particular be the case in embodiments of the invention relating to methods of treatment.

Thus, in one embodiment the radioactive label may be selected from the group consisting of ¹²⁵I and ¹³¹I.

For methods of treatment it is preferred that the radioactive label has cytotoxic properties. Thus, it is preferred that the radioactive label is sufficiently radioactive to be able to kill cells in the immediate vicinity. Thus it is preferred that at least 30 Gy, preferably at least 40 Gy, more preferably at least 50 Gy can be administered to the ccRCC tumour.

### Cytotoxic and Immunomodulatory Moiety

The agent for use in the methods of treatment of ccRCC according to the invention may be an SLC6A3 ligand linked to a cytotoxic or an immunomodulating moiety.

The cytotoxic moeity may for example be a cytotoxic drug used in treatment of cancer. Similarly, the immunomodulatory moeity may be an immunomodulatory drug used in the treatment of cancer.

Cytotoxic drugs or immunomodulatory drugs are well known to the skilled person.

Non limiting examples of cytotoxic drugs include the following:

### Alkylating Agents

Bendamustine
Busulfan
Carmustine
Chlorambucil
Cyclophosphamide
Dacarbazine
Ifosfamide
Melphalan
Procarbazine
Streptozocin
Temozolomide

### Anti-Metabolites

Asparaginase
Capecitabine
Cytarabine
5-Fluoro Uracil
Fludarabine
Gemcitabine
Methotrexate
Pemetrexed
Raltitrexed

### Anti-Tumour Antibiotics

Actinomycin D/Dactinomycin
Bleomycin
Daunorubicin
Doxorubicin
Doxorubicin (pegylated liposomal)
Epirubicin
Idarubicin
Mitomycin
Mitoxantrone

### Plant Alkaloids/Microtubule Inhibitors

Etoposide
Docetaxel
Irinotecan
Paclitaxel
Topotecan
Vinblastine
Vincristine
Vinorelbine

### DNA Linking Agents

Carboplatin
Cisplatin
Oxaliplatin

Non-limiting examples of immunomodulatory drugs (IMiDs) is thalidomide and its analogues, lenalidomide, pomalidomide and apremilast.

### Items

The invention may further relate to the following items:
1. An agent comprising or consisting of SLC6A3 ligand linked to a label (e.g. a radioactive label), a cytotoxic moiety or an immunomodulatory moiety for use in a method for diagnosis or treatment of clear cell renal cell carcinoma (ccRCC) in an individual.
2. The agent according to item 1, wherein the agent comprises or consists of a radioactively labelled SLC6A3 ligand for use in a method for diagnosis of ccRCC.
3. The agent according to item 2, wherein the method for diagnosis of ccRCC comprising administering said agent to said individual and detection of presence of the agent in the kidney of said individual, wherein the presence of said agent in the kidney of the individual is indicative of the presence of ccRCC.
4. The agent according to any one of the preceding items, wherein the method is for diagnosis of ccRCC or metastasized ccRCC.
5. The agent according to item 4, wherein the method comprises administering said agent to said individual and detection of the presence of the agent, wherein localised presence of said agent outside the central nervous system is indicative of presence of ccRCC or metastasized ccRCC in said individual.
6. The agent according to item 4, wherein the method comprises administering said agent to said individual and detection of the presence of said agent, wherein localised presence of said agent outside the kidney and outside the central nervous system is indicative of presence of metasized ccRCC.
7. The agent according to any one of items 2 to 6, wherein the presence of said agent in the lymph nodes is indicative of metastasized ccRCC.
8. The agent according to any one of the preceding items, wherein the agent is prepared for parenteral administration.
9. A method for diagnosing ccRCC in an individual, said method comprising the steps of
   a) administering an agent comprising or consisting of a SLC6A3 ligand linked to a radioactive label to an individual
   b) detecting the localised presence of said agent outside the central nervous system in said individual
   wherein the localised presence of said agent outside the central nervous system in the individual is indicative of the presence of ccRCC or metastasized ccRCC in said individual.
10. A method for diagnosing ccRCC in an individual, said method comprising detecting the localised presence of an SLC6A3 ligand linked to a radioactive label outside the central nervous system in said individual.
11. The method according to item 10, wherein said SLC6A3 ligand has been administered to said individual.
12. A method for determining the risk of presence of ccRCC in an individual, said method comprising the steps of
   a) administering an agent comprising or consisting of a SLC6A3 ligand linked to a radioactive label to an individual
   b) detecting the localised presence of said agent outside the central nervous system in said individual
   wherein the localised presence of said agent outside the central nervous system in the individual is indicative of the presence of ccRCC or metastasized ccRCC in said individual.
13. The method according to any one of items 9 to 12, wherein the presence of said agent in the kidney is indicative of presence of ccRCC in the individual.
14. The method according to any one of items 9 to 13, wherein the presence of said agent outside the kidney and central nervous system of said individual is indicative of presence of metastasized ccRCC.
15. The method according to any one of items 9 to 14, wherein the presence of said agent in the lymph nodes is indicative of metastasized ccRCC.
16. The method according to any one of the preceding items, wherein the individual is suffering from or has previously suffered from ccRCC.
17. The method according to any one of items 9 to 16, wherein said method comprises injecting said agent into said individual.
18. The agent or the method according to any one of the items 2 to 17, wherein the SLC6A3 ligand is a cocaine analogue.
19. The agent or the method according to any one of claims 2 to 5, wherein the SLC6A3 ligand contains or is linked to a radioactive label, which is selected from the group consisting of ¹²³I, ¹²⁵I, ¹¹C, ¹⁸F, ⁷⁶Br, ^{99m}Tc, ¹³N, ¹⁵O, ⁶⁸Ga, ⁸⁹Zr and ⁸²Rb.
20. The agent or the method according to any one of items 2 to 18, wherein the SLC6A3 ligand contains or is linked to a radioactive label, which is ¹²³I.
21. The agent according to item 1, wherein the agent comprises or consists of SLC6A3 ligand linked to a radioactive label, a cytotoxic moiety or an immunomodulatory moiety for use in a method of treatment of clear cell renal cell carcinoma (ccRCC) in an individual in need thereof.
22. A method of treatment of ccRCC in an individual in need thereof, said method comprising administering an agent comprising or consisting of a SLC6A3 ligand linked to a radioactive label, a cytotoxic moiety or an immunomodulatory moiety to said individual in a pharmaceutically effective amount.
23. The agent or the method according to any one of the items 21 to 22, wherein the SLC6A3 ligand is an amphetamine analogue.
24. The agent or the method according to any one of the items 21 to 22, wherein the SLC6A3 ligand is a cocaine analogue.
25. The agent or the method according to any one of the preceding items, wherein the SLC6A3 ligand is not capable of passing the blood-brain barrier.
26. The agent or the method according to any one of the preceding items, wherein the SLC6A3 ligand has a Ki in respect of SLC6A3 of at the most 100 nM.
27. The agent or the method according to any one of the preceding items, wherein the SLC6A3 ligand has a Kd in respect of SLC6A3 of at the most 10,000 nM.
28. The agent or the method according to any one of the preceding items, wherein the SLC6A3 ligand has a Ki in respect of SERT, which is at least 10 times higher than the Ki in respect of SLC6A3.
29. The agent or the method according to any one of the items 22 to 28, wherein the SLC6A3 ligand contains or is linked to a radioactive label, which is selected from the group consisting of ¹²⁵I and ¹³¹I.
30. The agent or the method according to any one of the preceding items, wherein the ccRCC is localised ccRCC.
31. The agent or the method according to any one of the preceding items, wherein the ccRCC is metastasized ccRCC.
32. The agent or the method according to any one of the preceding items, wherein the SLC6A3 ligand is a tropane derivative or a nortropane derivative.
33. The agent or the method according to any one of the preceding items, wherein the SLC6A3 ligand is a compound of formula I:

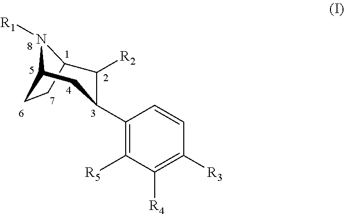

wherein
R₁ is C₁₋₆-alkyl, C₂₋₆-alkenyl, phenyl or benzyl, wherein said C₁₋₆-alkyl, C₂₋₆-alkenyl phenyl or benzyl optionally may be substituted with one or more halogen;
R₂ is CO₂—R₆, C═N—O—R₆ or heteroaryl-R₆, wherein R₆ is C₁₋₆-alkyl, C₂₋₆-alkenyl, phenyl or benzyl, wherein said C₁₋₆-alkyl, C₂₋₆-alkenyl phenyl or benzyl optionally may be substituted with one or more halogen;
R₃ is C₁₋₃-alkyl, halogen or heteroaryl, wherein said C₁₋₃-alkyl optionally may be substituted with halogen; and
R₄ and R₅ individually are H, C₁₋₃-alkyl or halogen, wherein said C₁₋₃-alkyl optionally may be substituted with halogen.
34. The agent or the method according to item 33, wherein
R₁ is C₁₋₆-alkyl or C₂₋₆-alkenyl, wherein said C₁₋₆-alkyl or C₂₋₆-alkenyl optionally may be substituted with one or more halogen;
R₂ is CO₂—R₆, wherein R₆ is C₁₋₆-alkyl or C₂₋₆-alkenyl, wherein said C₁₋₆-alkyl or C₂₋₆-alkenyl optionally may be substituted with one or more halogen;
R₃ is C₁₋₃-alkyl or halogen, wherein said C₁₋₃-alkyl optionally may be substituted with halogen; and
R₄ and R₅ individually are H, C₁₋₃-alkyl or halogen, wherein said C₁₋₃-alkyl optionally may be substituted with halogen.
35. The agent or the method according to item 33, wherein
R₁ is C₁₋₄-alkyl or C₂₋₄-alkenyl, wherein said C₁₋₄-alkyl or C₂₋₄-alkenyl optionally may be substituted with halogen;
R₂ is CO₂—R₆, wherein R₆ is C₁₋₄-alkyl, wherein said C₁₋₄-alkyl optionally may be substituted with halogen;
R₃ is C₁₋₃-alkyl or halogen, wherein said C₁₋₃-alkyl optionally may be substituted with halogen; and
R₄ and R₅ individually are H, C₁₋₃-alkyl or halogen, wherein said C₁₋₃-alkyl optionally may be substituted with halogen.
36. The agent or the method according to any one of items 33 to 35, wherein at least one of R₁, R₂, R₃, R₄ or R₅ comprises halogen.
37. The agent or the method according to any one of items 33 to 36, wherein at least one of R₁, R₂, R₃, R₄ or R₅ comprise radioactive isotope of a halogen.
38. The agent or the method according to any one of the preceding items, wherein the SLC6A3 ligand linked to a radioactive label is selected from the group consisting of I-FMIP, β-CFT (WIN 35,428), β-CIT (RTI-55), β-CCT (RTI-31), β-CMT (RTI-32), FECNT, β-CCT-FP (β-FPCT), β-CBT, β-FECT (β-FE-CCT), β-FETT (β-FE-CMT), β-FIPCT (β-FiP-CCT), β-CFT-FE, β-CFT-FP, β-CBT-FE, β-CIT-FP (FP-CIT), β-CMT-FP (FP-CMT), β-CBT-FP (FP-CBT), β-CDCT, β-IP-CIT (RTI-121), NS-2214 (BMS-204756), β-C IT-FE, β-CpFMT, β-CmFMT, β-CoFMT (o-FWIN), β-CPPIT, FCT, E-IACFT (Altropane), MCL301, MCL322, FE@CIT, FBCINT, FE-3FPT and PE2I.
39. The agent or the method according to any one of the preceding items, wherein the SLC6A3 ligand linked to a radioactive label is selected from the group consisting of ¹²³I-FMIP. [¹¹C]-2β-carbomethoxy-3β-Itropane, 2β-carbomethoxy-3β-(4-fluorophenyl)-N-((E)-3-iodo-prop-2-enyl)tropane, [¹²³I]-(1R)-2-β-Carbomethoxy-3-β-(4-iodophenyl)-tropane, [¹²³I] N-ω-fluoropropyl-2β-carbomethoxy-3β-(4-iodophenyl)-nortropane, [^{99m}Tc]technetium [2-[[2-[[[3-(4-chlorophenyl)-8-methyl-8-azabicyclo-[3.2.1]oct-2-yl]-methyl](2-mercaptoethyl)amino]-ethyl]amino]ethanethiolato(3−)-N2,N2′,S2,S2′]oxo-[1R-(exoexo)], [¹²³I]-2β-carbomethoxy-3β-(4-fluorophenyl)-N-(1-iodoprop-1-en-3-yl)-ortropane, and [¹²³I]—N-(3-iodopropen-2-yl)-2-carbomethoxy-3beta-(4-chlorophenyl)-tropane.
40. The agent or the method according to any one of the preceding items, wherein the SLC6A3 ligand linked to a radioactively label is [¹²³I] N-ω-fluoropropyl-2β-carbomethoxy-3β-(4-iodophenyl)nortropane.
41. The agent or the method according to any one of items 1 to 31, wherein the SLC6A3 ligand is a compound of formula IIIa or IIIb:

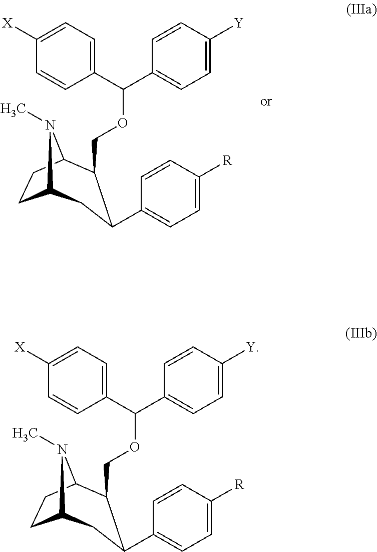

42. The agent or the method according to item 41, wherein the X and Y is H or halogen and R is halogen.
43. The agent or the method according to any one of items 1 to 31, wherein the SLC6A3 ligand is a compound of the formula IV:

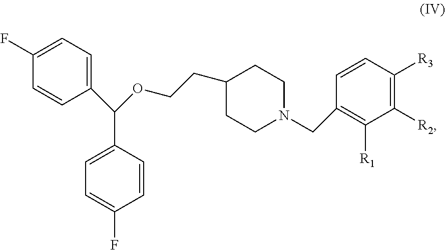

wherein
R₁, R₂ and R₃ individually may be selected from the group consisting of —H, halogen, C₁₋₃-alkyl, C₁₋₃-alkyl substituted with one or more halogen, —CN, and NO₂.
44. The agent or the method according to item 43, wherein at least two of R₁, R₂ and R₃ are H, and the third is selected from the group consisting of halogen, C₁₋₃-alkyl, C₁₋₃-alkyl substituted with one or more halogen, —CN, and NO₂.
45. The agent or the method according to any one of items 43 and 44, wherein at least one of R₁, R₂ and R₃ is halogen.
46. The agent or the method according to any one of items 1 to 31, wherein the SLC6A3 ligand is a compound of the formula V:

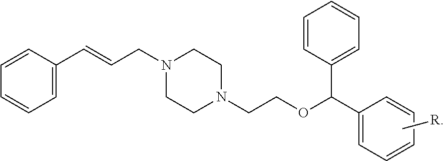

47. The agent or the method according to item 46, wherein R is selected from the group consisting of —H, halogen, —NH2, —NCS, .NO2, and maleimide.
48. The agent or the method according to any one of items 1 to 31, wherein the SLC6A3 ligand is a compound of the formula VI:

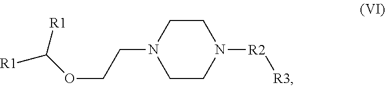

wherein
R₁ is aryl optionally substituted with one or more -halogen, —NH₂, —NCS, —NO₂, or Maleimid-1-yl;
R₂ is C₁₋₃-alkyl, C₁₋₃ alkenyl, —CH₂-cyclopropyl, or —H₂C—C≡C—; and
R₃ is —H, aryl, heteroaryl, bicyclic aromatic ring or heterocyclic ringsystem, which can be partially or fully saturated, wherein any of the aforementioned optionally may be substituted with halogen, —OH, alkoxy or oxy.
49. The agent or the method according to item 48, wherein R₁ is phenyl optionally substituted with one or more -halogen, —NH₂, —NCS, —NO₂, or Maleimid-1-yl.
50. The agent or the method according to any one of items 48 and 49, wherein R₃ is phenyl.
51. The agent or the method according to any one of items 1 to 31, wherein the SLC6A3 ligand is a compound of the formula VII:

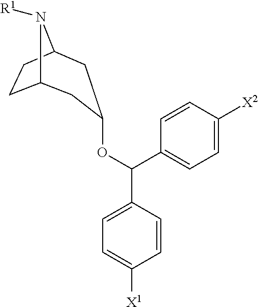

wherein
R₁ is H or L-B; wherein
   L is C₁₋₆-alkyl, or —CH₂CH₂—Y—CH₂CH₂—, wherein
      Y is —NH— or —O—; and
   B is —OH, halogen, aryl, heterocyclyl, —H, —OR₃, or NHR₂, wherein
      R₂ is —CO-aryl or —CO-heteroaryl; and
   X₁ and X₂ independently are —H, C₁₋₆-alkyl, halogen, —OH, —O—C₁₋₆-alkyl, —NH₂, —NR₃R₄, wherein
      R₃ and R₄ independently are H, C₁₋₆-alkyl, —CN or NO₂.
52. The agent or the method according to any one of items 1 to 31, wherein the SLC6A3 ligand is a compound of the formula VIII:

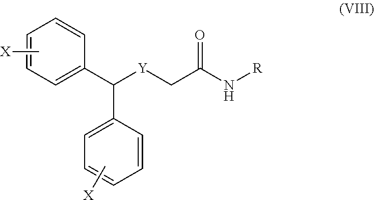

wherein
Y is —S— or SO;
X is independently —H, C₁₋₆-alkyl, halogen or CN; and
R is C₁₋₆ alkyl or cycloalkyl, optionally substituted with aryl.
53. The agent or the method according to any one of items 1 to 31, wherein the SLC6A3 ligand is a compound of the formula IX:

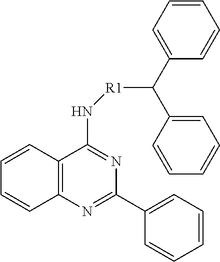

wherein
R₁ may be C₁₋₃ alkyl.
54. The agent or the method according to any one of items 1 to 31, wherein the SLC6A3 ligand is selected from GBR12909, GBR12935, 1-piperazinepropanamine, 4-[2-[4-azido-3-(iodo-)phenyl]ethyl]-N,N-bis(4-fluorophenyl)-9(Cl) and FMIP.
55. The agent or the method according to any one of items 2 to 20, wherein the presence of the agent in the kidney of said individual is detected by positron emission tomography (PET) or by single-photon emission computed tomography (SPECT).
56. Use of an SLC6A3 ligand linked to a radioactive label, a cytotoxic moiety or an immunomodulatory moiety for use in the preparation of a medicament for use in the diagnosis or treatment of clear cell renal cell carcinoma (ccRCC) in an individual.
57. Use according to item 56, wherein the SLC6A3 ligand is as defined in any one of the preceding items.

### EXAMPLES

### Example 1

### Expression of Functional SLC6A3

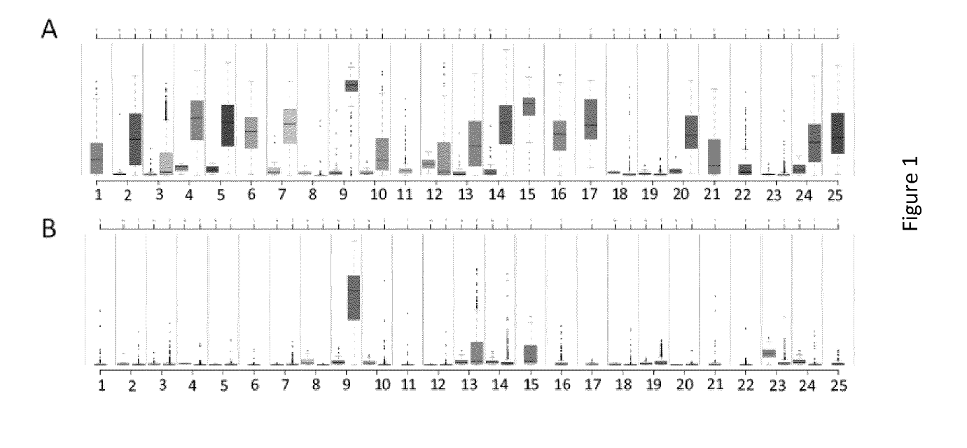

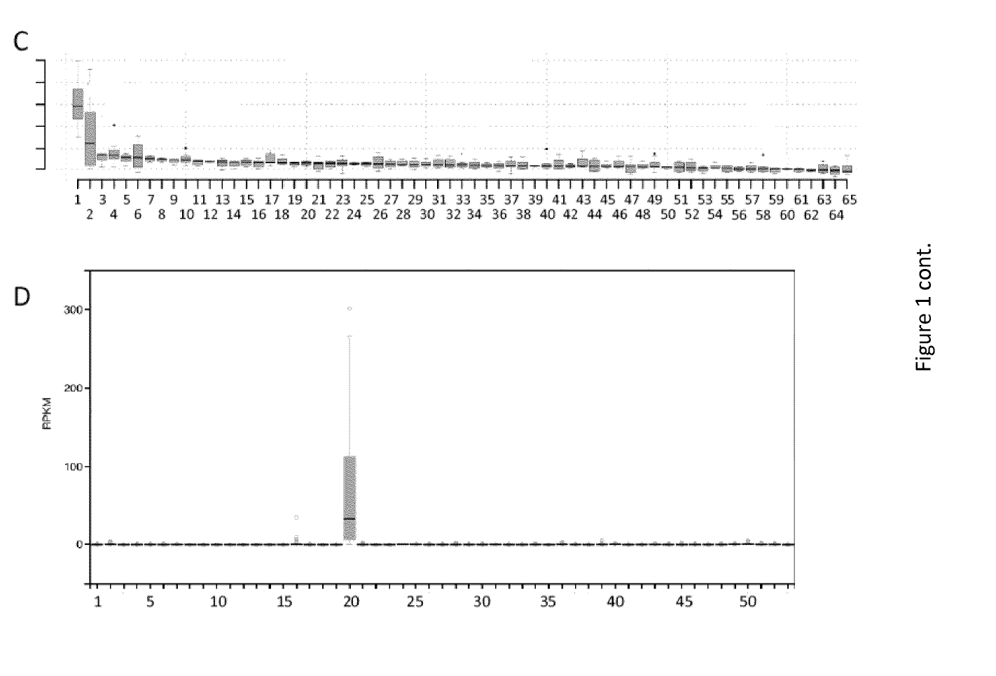

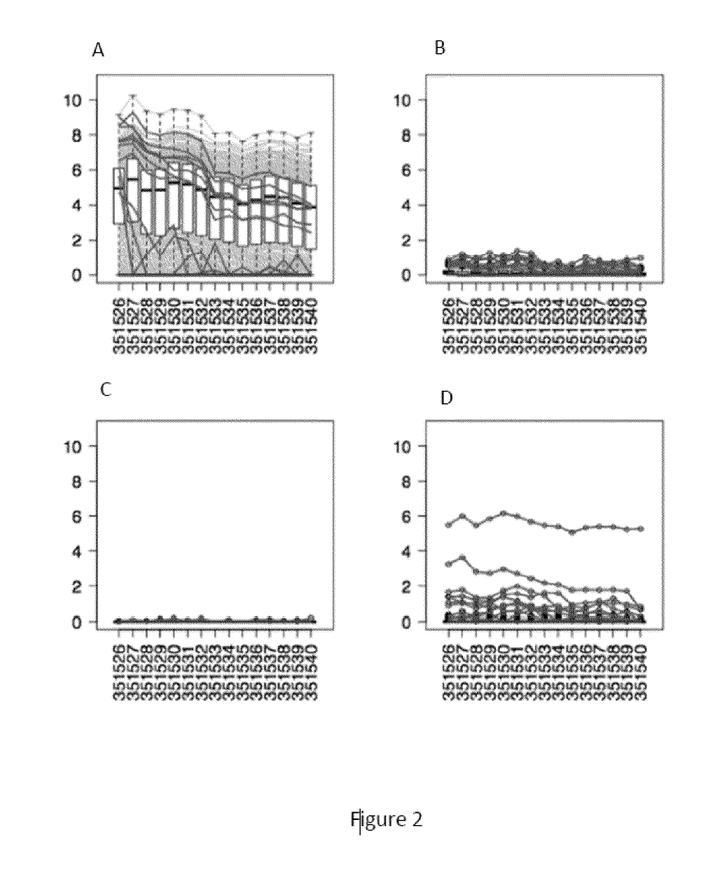

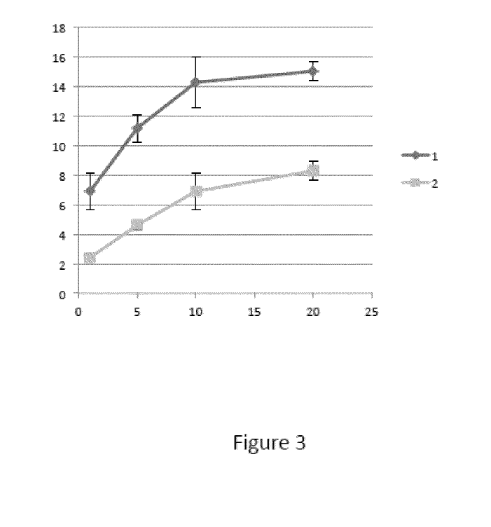

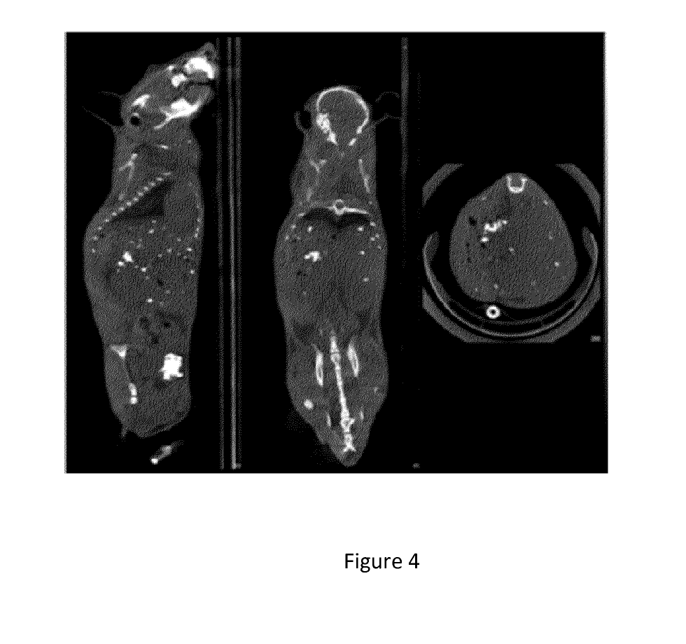

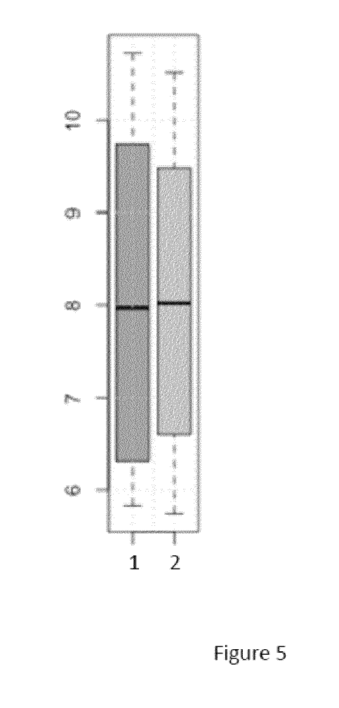

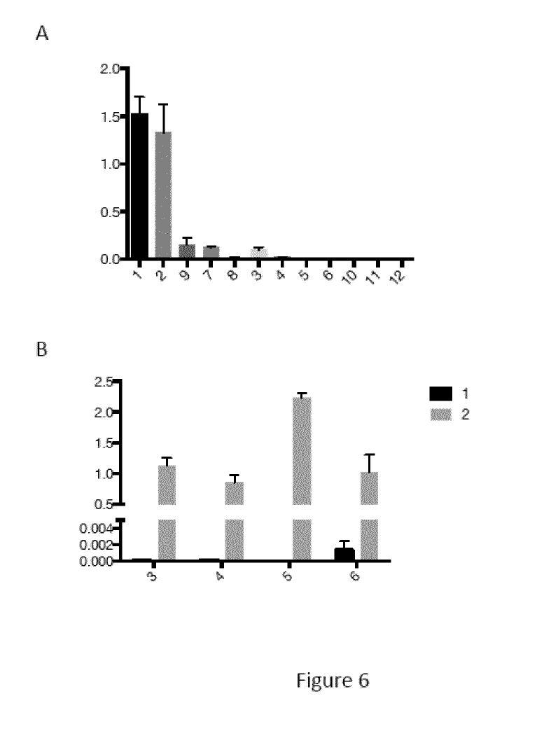

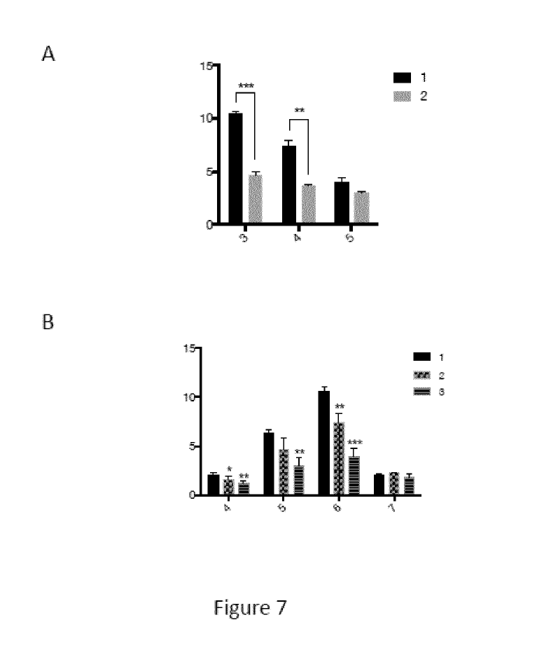

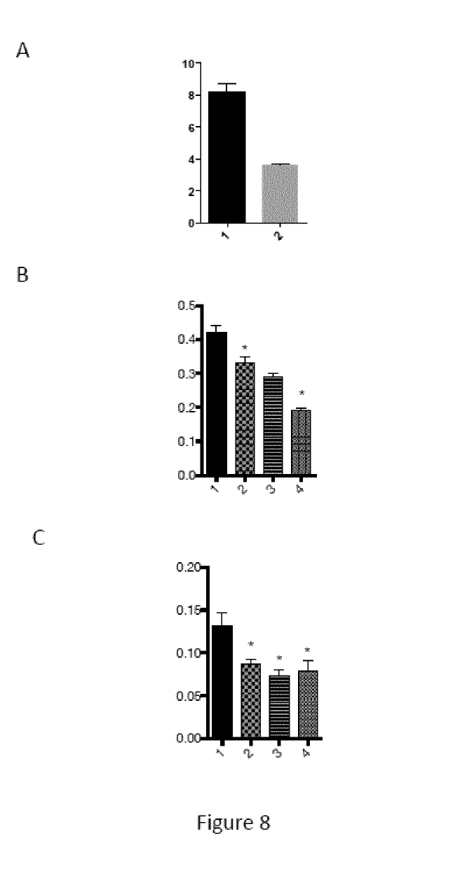

A comprehensive bioinformatics platform for in depth analyses of the recently published TOGA (The Cancer Genome Atlas) data set comprising more than 500 ccRCC and a few hundred other renal malignancies was established. The data sets contained clinical information, genomic characterization data, and high level sequence analysis of the tumor genomes. We here demonstrate that SLC6A3 showed an exceptionally high expression of mRNA in ccRCC, both in the primary tumor and its metastases, while no other analyzed tumor types displayed appreciable expression of the gene (see FIG. 1 and FIG. 5).

The analysis is based on RNA sequence data with gene specific reads through the 15 exons of SLC6A3 based on analyses of The Cancer Genome Atlas cohort, and it shows that the sequence reads distribute over all exons of the gene (see FIG. 2). This demonstrates that the entire gene is transcribed in ccRCC.

SLC6A3 (also known as Dat1) is a dopamine transporter normally expressed primarily in dopaminergic neurons in the central nervous system (see FIG. 1C-D). Its main function is to clear the synapses from released dopamine, for sequestration into vesicles for later release.

A series of experiments on both established and primary ccRCC cells using [³H]-dopamine were performed. Uptake experiments were performed in cells cultivated in 12-well plates at 37° C. The cells of interest were seeded at 80.000 cells/well 48 h before the uptake experiment was performed. The wells were washed in 1×PBS, followed by a wash in 1 ml of assay buffer (Hanks Balanced Salt Solution (HBSS) with CaCl₂ and MgCl₂, without phenol red supplemented with 10 mM HEPES, pH 7.4) at 37° C. and then incubated with 0.5 ml of buffer for 5 min. The wells were thereafter incubated with assay buffer containing 7 nM [³H]dopamine (56.8 Ci/mmol) for the following time points 1, 5, 10 and 20 min. For competition experiments cells were incubated with assay buffer containing 7 nM [³H]dopamine (56.8 Ci/mmol) supplemented with competitive unlabelled dopamine at 0 uM, 2 uM or 20 uM for 5 min. For specific SLC6A3 inhibition, cells were pre incubated with inhibitor GBR12935 (30 nM) or GBR12909 (10-90 nM) for one hour and uptake was thereafter performed by incubation with assay buffer containing 7 nM [³H]dopamine (56.8 Ci/mmol) in the presence of GBR12935 (30 nM) or GBR12909 (10-90 nM). All uptake experiments were stopped by removing the uptake buffer and adding 1 ml of ice-cold assay buffer to each well and immediately aspirating the buffer and washing three additional times with 1 ml of ice-cold buffer. The incorporated radioactivity was extracted by solubilising the cells with 1% Triton-X for 1 h at R.T, 6 ml of scintillation fluid was thereafter added. The amount of radioactivity was measured with a Tri-carb 2810TR liquid scintillation analyzer (Perkin Elmer).

The experiments demonstrates that ccRCC cell lines take up [H³]dopamine and show a strong correlation between expression level of SLC6A3 and dopamine uptake. Also, primary ccRCC cells display an enhanced uptake of dopamine compared to primary normal cortex cell (see FIG. 3 and FIG. 7A. Additionally, primary ccRCC tumor cells show decreased uptake in response to competitive unlabelled dopamine (FIG. 7A-B). Amongst the established ccRCC cell lines investigated, all but two (KMRC-3 and SNU-349), express relatively low levels of SLC6A3 (see FIG. 6A). In contrast, all investigated primary ccRCC tumors and short term primary cell cultures express high levels of the transporter (FIG. 6B).

### Example 2

The regulation and function of SLC6A3 has been widely studied in relation to its function in dopaminergic neurons and its role in neurological disease such as Parkinson's disease. Several compounds, including cocaine and amphetamine, exert their effects through interference with SLC6A3 and hence the level of dopamine in the synaptic cleft.

It was tested whether the approved radiopharmaceutical drug, Ioflupane (¹²³I) (GE Healthcare) could be useful for detection of SLC6A3 in the kidney. Ioflupane (¹²³I) is used for the diagnosis of Parkinson's disease, where Ioflupane (¹²³I) is used to detect function of SLC6A3 in the synaptic cleft. The function of SLC6A3 is highly dependent on a Na⁺ gradient, with high extracellular concentration and lower Na⁺ concentration mediates decreased uptake of [3H]-dopamine. Thus, due to the difference in microenvironment in ccRCC tumors versus the one of the dopaminergic synaptic cleft, it was unclear whether Ioflupane (¹²³I) would be useful in a kidney environment. The chemical name of Ioflupane (¹²³I) is [¹²³I] N-ω-fluoropropyl-2β-carbomethoxy-3β-(4-iodophenyl)nortropane and the structure is provided herein in Table 1.

ccRCC cell line KMRC-3 cells were injected orthotopically in mice, 2*10⁶ cells in 40 ul PBS. After 13 weeks the mice were injected with 5.36 MBq ¹²³I-Ioflupan and analysed by SPECT/CT imaging after 1 h and 35 min. Analyses of the experiments indicate that the orthotopically injected kidney cells clearly display enhanced uptake of Ioflupane (¹²³I) when analyzed using SPECT/CT, while the contralateral kidney lacking injected tumor cells show no enhanced uptake of Ioflupane (¹²³I) (see FIG. 4).

In addition, [³H]-dopamine uptake experiments were performed in the presence of GBR12935 and GBR12909, cocaine analogues that are specific inhibitors of SLC6A3. Using these inhibitors at low concentrations could clearly diminish the amount of [³H]-dopamine taken up by ccRCC cells (see FIG. 8 A-C).

Accordingly, both GBR12935 and GBR12909 can functionally interact with ccRCC cells expressing SLC6A3.

### Example 3

Analysis of expression of SLC6A3 was investigated in 16 matched primary ccRCC tumors and metastasis (Lopez-Lago et al., Cancer Res 70(23) 2010).

As shown in FIG. 5, SLC6A3 is expressed both in the primary ccRCC and in the metastasis.

### REFERENCES

Boos et al., “Structure-activity relationships of substituted N-benzyl piperidines in the GBR series: Synthesis of 4-(2-(bis(4-fluorophenyl)methoxy)ethyl)-1-(2-trifluoromethylbenzyl piperidine, an allosteric modulator of the serotonin transporter”, Bioorganic & Medicinal Chemistry (2006), 14:3967-3973,
De Bruyne et al., “Synthesis, radiosynthesis and in vivo evaluation of [123I]-4-(2-(bis(4-fluorophenyl)methoxy)ethyl)-1-(4-iodobenzyl)piperidine as a selective tracer for imaging the dopamine transporter” J. Label Compd. Radiopharm (2009), 52 304-311
De Bruyne et al., “In vivo evaluation of [123I]-4-(2-(bis(4-fluorophenyl)methoxy)ethyl)-1-(4-iodobenzyl)piperidine, an iodinated SPECT tracer for imaging the P-gp transporter”, Nuclear Medicine and Biology (2010), 37:469-477
J Chen and W A Weiss “Alternative splicing in cancer: implications for biology and therapy” Oncogene (2015), 34:1-14
Greiner E et al., “Structure-activity relationship studies of highly selective inhibitors of the dopamine transporter: N-benzylpiperidine analogues of 1-[2-[bis(4-fluorophenyl)methoxy]ethyl]-4-(3-phenylpropyl)piperazine” J. Med. Chem. (2003), 46:1465-1469.
Hong et al., “2-Substituted 3b-Aryltropane Cocaine Analogs Produce Atypical Effects without Inducing Inward-Facing Dopamine Transporter Conformations” J Pharmacol Exp Ther (2016), 356:624-634
Hout P et al., “Monoamine Reuptake Inhibitors in Parkinson's Disease”, Parkinson's Dis (2015), 2015:609428
López-Lago M A et al., “Genomic deregulation during metastasis of renal cell carcinoma implements a myofibroblast-like program of gene expression”. Cancer Res (2010), 70:9682-92
Skubitz K M, Skubitz A P “Differential gene expression in renal-cell cancer”. J Lab Clin Med. (2002) 140:52-64.
Sonders M S, et al., “Multiple ionic conductances of the human dopamine transporter: the actions of dopamine and psychostimulants”. J. Neurosci. (1997), 17:960-74.
Torres G E et al., “Plasma membrane monoamine transporters: structure, regulation and function” Nat. Rev. Neurosci (2003), 4:13-25
Wheeler D D, et al., “A model of the sodium dependence of dopamine uptake in rat striatal synaptosomes”. Neurochem. Res. (1993), 18:927-936.

1. A method for diagnosis or treatment of clear cell renal cell carcinoma (ccRCC) comprising administration of an agent comprising or consisting of SLC6A3 ligand linked to a label, a cytotoxic moiety or an immunomodulatory moiety to an individual in need thereof.

2. The method according to claim 1, wherein the method is a method for diagnosis of ccRCC, wherein the agent comprises or consists of a radioactively labelled SLC6A3 ligand.

3. The method according to claim 2, wherein the method comprises administering said agent to said individual and detection of the presence of the agent, wherein localised presence of said agent outside the central nervous system is indicative of presence of ccRCC or metastasized ccRCC in said individual.

4. The method according to claim 1, wherein the method comprises administering said agent to said individual and detection of the presence of said agent, wherein localised presence of said agent outside the kidney and outside the central nervous system is indicative of presence of metastasized ccRCC.

5. A method for diagnosing ccRCC in an individual, said method comprising the steps of
a) administering an agent comprising or consisting of a SLC6A3 ligand linked to a radioactive label to an individual
b) detecting the localised presence of said agent outside the central nervous system in said individual
wherein the localised presence of said agent outside the central nervous system in the individual is indicative of the presence of ccRCC or metastasized ccRCC in said individual.

6. The method according to claim 2, wherein the SLC6A3 ligand contains or is linked to a radioactive label, which is selected from the group consisting of ¹²³I, ¹²³I, ¹¹C, ¹⁸F, ⁷⁶Br, ^{99m}Tc, ¹³N, ¹⁵O, ⁶⁸Ga, ⁸⁹Zr and ⁸²Rb.

7. The method according to claim 2, wherein the SLC6A3 ligand contains or is linked to a radioactive label, which is ¹²³I.

8. The method according to claim 1, wherein the agent comprises or consists of SLC6A3 ligand linked to a radioactive label, a cytotoxic moiety or an immunomodulatory moiety for use in a method of treatment of clear cell renal cell carcinoma (ccRCC) in an individual in need thereof.

9. The method according to claim 1, wherein the SLC6A3 ligand is an amphetamine analogue.

10. The method according to claim 1, wherein the SLC6A3 ligand is a cocaine analogue.

11. The method according to claim 1, wherein the SLC6A3 ligand has a Ki in respect of SLC6A3 of at the most 100 nM, and/or a Kd in respect of SLC6A3 of at the most 10,000 nM.

12. The method according to claim 1, wherein the SLC6A3 ligand is a tropane derivative or a nortropane derivative.

13. The method according to claim 1, wherein the SLC6A3 ligand is a compound of formula I: 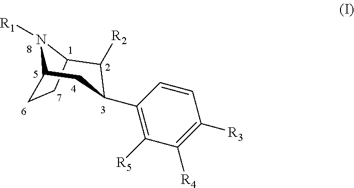
wherein
R₁ is C₁₋₆-alkyl, C₂₋₆-alkenyl, phenyl or benzyl, wherein said C₁₋₆-alkyl, C₂₋₆-alkenyl phenyl or benzyl optionally may be substituted with one or more halogen;
R₂ is CO₂—R₆, C═N—O—R₆ or heteroaryl-R₆, wherein R₆ is C₁₋₆-alkyl, C₂₋₆-alkenyl, phenyl or benzyl, wherein said C₁₋₆-alkyl, C₂₋₆-alkenyl phenyl or benzyl optionally may be substituted with one or more halogen;
R₃ is C₁₋₃-alkyl, halogen or heteroaryl, wherein said C₁₋₃-alkyl optionally may be substituted with halogen; and
R₄ and R₅ individually are —H, C₁₋₃-alkyl or halogen, wherein said C₁₋₃-alkyl optionally may be substituted with halogen.

14. The method according to claim 1, wherein the SLC6A3 ligand is a compound of formula IIIa or IIIb:

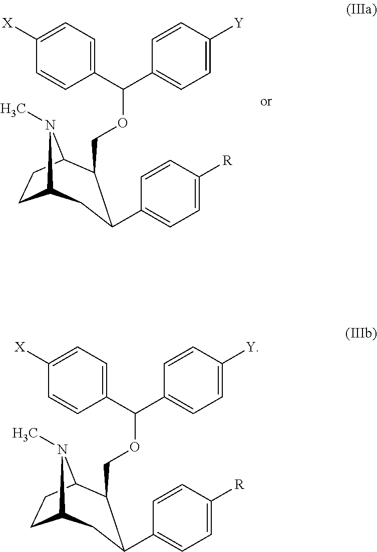

15. The method according to claim 1, wherein the SLC6A3 ligand is a compound of the formula IV: 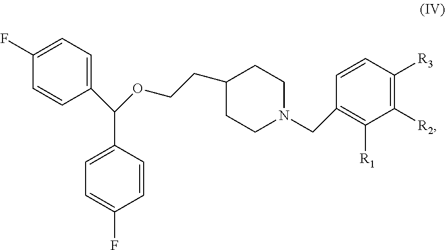
wherein
R₁, R₂ and R₃ individually may be selected from the group consisting of —H, halogen, C₁₋₃-alkyl, C₁₋₃-alkyl substituted with one or more halogen, —CN, and NO₂.

16. The method according to claim 1, wherein the SLC6A3 ligand is a compound of the formula V:

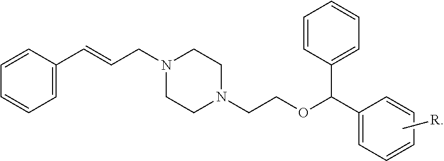

17. The method according to claim 1, wherein the SLC6A3 ligand is a compound of the formula VI: 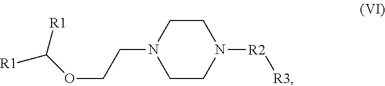
wherein
R₁ is aryl optionally substituted with one or more -halogen, —NH₂, —NCS, —NO₂, or Maleimid-1-yl;
R₂ is C₁₋₃-alkyl, C₁₋₃ alkenyl, —CH₂-cyclopropyl, or —H₂C—C≡C—; and
R₃ is —H, aryl, heteroaryl, bicyclic aromatic ring or heterocyclic ringsystem, which can be partially or fully saturated, wherein any of the aforementioned optionally may be substituted with halogen, —OH, alkoxy or oxy.

18. The method according to claim 1, wherein the SLC6A3 ligand is a compound of the formula VII: 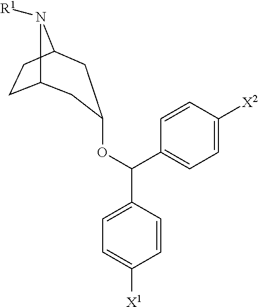
wherein
R₁ is —H or L-B; wherein
L is C₁₋₆-alkyl, or —CH₂CH₂—Y—CH₂CH₂—, wherein
Y is —NH— or —O—; and
B is —OH, halogen, aryl, heterocyclyl, —H, —OR₃, or NHR₂, wherein
R₂ is —CO-aryl or —CO-heteroaryl; and
X₁ and X₂ independently are —H, C₁₋₆-alkyl, halogen, —OH, —O—C₁₋₆-alkyl, —NH₂, —NR₃R₄, wherein
R₃ and R₄ independently are —H, C₁₋₆-alkyl, —CN or NO₂.

19. The method according to claim 1, wherein the SLC6A3 ligand is a compound of the formula VIII: 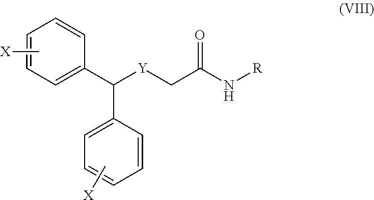
wherein
Y is —S— or SO;
X is independently —H, C₁₋₆-alkyl, halogen or CN; and
R is C₁₋₆ alkyl or cycloalkyl, optionally substituted with aryl.

20. The method according to claim 1, wherein the SLC6A3 ligand is a compound of the formula IX: 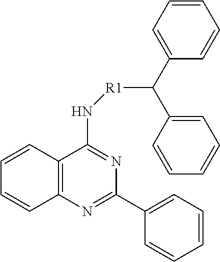
wherein
R₁ may be C₁₋₃ alkyl.

21. The method according to claim 1, wherein the SLC6A3 ligand is selected from GBR12909, GBR12935, 1-piperazinepropanamine, 4-[2-[4-azido-3-(iodo-)phenyl]ethyl]-N,N-bis(4-fluorophenyl)-9(Cl) and FMIP.

22. The method according to claim 2, wherein the SLC6A3 ligand linked to a radioactive label is selected from the group consisting of β-CFT (WIN 35,428), β-CIT (RTI-55), β-CCT (RTI-31), β-CMT (RTI-32), FECNT, β-CCT-FP (β-FPCT), β-CBT, β-FECT (β-FE-CCT), β-FETT (β-FE-CMT), β-FIPCT (β-FiP-CCT), β-CFT-FE, β-CFT-FP, β-CBT-FE, β-CIT-FP (FP-CIT), β-CMT-FP (FP-CMT), β-CBT-FP (FP-CBT), β-CDCT, β-IP-CIT (RTI-121), NS-2214 (BMS-204756), β-C IT-FE, β-CpFMT, β-CmFMT, β-CoFMT (o-FWIN), β-CPPIT, FCT, E-IACFT (Altropane), MCL301, MCL322, FE@CIT, FBCINT, FE-3FPT, PE2I, [¹¹C]-2β-carbomethoxy-3β-Itropane, 2β-carbomethoxy-3β-(4-fluorophenyl)-N-((E)-3-iodo-prop-2-enyl)tropane, [¹²³I]-(1R)-2-β-Carbomethoxy-3-β-(4-iodophenyl)-tropane, [¹²³I] N-ω-fluoropropyl-2β-carbomethoxy-3β-(4-iodophenyl)-nortropane, [^{99m}Tc]technetium [2-[[2-[[[3-(4-chlorophenyl)-8-methyl-8-azabicyclo-[3.2.1]oct-2-yl]-methyl](2-mercaptoethyl)amino]-ethyl]amino]ethanethiolato(3−)-N2,N2′,S2,S2′]oxo-[1R-(exoexo)], [¹²³I]-2β-carbomethoxy-3β-(4-fluorophenyl)-N-(1-iodoprop-1-en-3-yl)-ortropane, and [¹²³I]—N-(3-iodopropen-2-yl)-2-carbomethoxy-3beta-(4-chlorophenyl)-tropane.
